(19) United States
(12) Patent Application Publication
(10) Pub. No.: US 2006/0062848 A1
(43) Pub. Date: Mar. 23, 2006

(54) Formulation comprising itraconazole

(75) Inventors: Caroline German, Manchester (GB); Raymond Sloan, Bradford (GB)

Correspondence Address: NEKTAR THERAPEUTICS, 150 INDUSTRIAL ROAD, SAN CARLOS, CA, 94070, US

(73) Assignee: Nektar Therapeutics UK Limited, Bradford (GB)

(21) Appl. No.: 11/228,541

(22) Filed: Sep. 16, 2005

Related U.S. Application Data
(60) Provisional application No. 60/611,102, filed on Sep. 17, 2004.

(51) Int. Cl. A61K 31/496; A61K 9/20

(52) U.S. Cl. 424/464; 514/254.07

(57) ABSTRACT

Formulations of azole antifungals such as itraconazole and particularly formulations, co-formulations and compositions of itraconazole with one or more oligomeric and/or polymeric excipients are disclosed. Methods for preparation of the formulations, co-formulations and compositions include co-precipitating the two materials from a common solvent or solvent mixture using a compressed (typically supercritical or near-critical) fluid anti-solvent as in the GAS (Gas Anti-Solvent) precipitation method. The formulations, co-formulations, compositions, methods of making and methods of delivering, are useful as pharmaceutical compositions and in medical treatment by virtue of their at least parity, preferably improved or enhanced solubility or dissolution characteristics, resulting in at least parity, preferably improved or enhanced bioavailability and/or pharmacokinetics.

### RELATED APPLICATION

This application relates to U.S. Provisional Application No. 60/611,102 filed Sep. 17, 2004, from which priority is claimed under 35 USC §119(e), and which is incorporated herein in its entirety.

### BACKGROUND OF THE INVENTION

1. Field of the Invention

The present invention relates to formulations of azole antifungals such as itraconazole and particularly to co-formulations of itraconazole with excipients, to methods for their preparation, pharmaceutical compositions comprising them and their use in medical treatment. The present invention relates more particularly to co-formulations of itraconazole with one or more oligomeric and/or polymeric excipients, and to methods of making and methods of delivering, which result in improved or enhanced solubility or dissolution characteristics, resulting in improved or enhanced bioavailability and/or pharmacokinetics.

2. Description of Related Art

Itraconazole, (±) cis-4-[4-[4-[4-[[2-(2,4-dichlorphenyl)-2-(1H-1,2,4-triazol-1-ylmethyl)-1,3-dioxolan-4-yl]methoxy]phenyl]-1-piperazinyl]phenyl]-2,4-dihydro-2-(1-methylpropyl)-3H-1,2,4-triazol-3-one, has the following chemical structure:

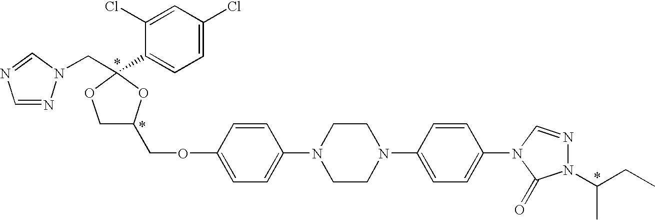

Itraconazole can exist as a 1:1:1:1 racemic mixture of four diastereomers (two enantiomer pairs), each possessing three chiral centres. Itraconazole has a molecular weight of 705.6 g/mole and its empirical formula is: C₃₅H₃₈Cl₂N₈O₄.

Itraconazole is a triazole anti-fungal agent with broad spectrum activity against a wide range of systemic fungal infections (blastomycosis, histoplasmosis, aspergillosis, oral candidiasis) and is an effective treatment for fungal infections in both fingernails and toenails (onychomycosis). Itraconazole has found particular application in the treatment of immuno-compromised patients with fungal infections, especially fungal infections in patients undergoing chemotherapy, or afflicted with AIDS and AIDS-related conditions, or organ transplant recipients.

In common with other azole antifungal agents, itraconazole is only very sparingly soluble in aqueous media, possessing an aqueous solubility of 1 ng/ml at neutral pH & 6 μg/ml at pH 1. Moreover, itraconazole itself possesses a relatively low potency, necessitating relatively large dosages, on the order of 200-400 mg. Dosing durations of existing itraconazole formulations can vary from one week to three months or more, depending upon the indication. As a consequence, the development of pharmaceutical compositions of itraconazole having acceptable solubility and/or dissolution characteristics, and consequent bioavailability (BAV), especially when intended for oral or intravenous administration, has presented considerable challenges.

Approaches to improving the solubility of itraconazole which have been described previously include complexing the drug with cyclodextrins, or derivatives thereof as discussed, for example, in U.S. Pat. No. 4,727,064, U.S. Pat. No. 5,707,975 and U.S. Pat. No. 6,407,079, the contents of which are incorporated herein by reference in their entireties. Orally-delivered forms of itraconazole are currently marketed by Janssen Pharmaceuticals under the trade name Sporanox™. In one form, the itraconazole is formulated for delivery by capsule. In another, the active is formulated as an oral solution, with a cyclodextrin complex. Oral solutions, have however, been shown to result in poor patient compliance. An intravenous formulation of itraconazole in the form of a complex with hydroxypropyl-β-cyclodextrin (Ortho Biotech) is also commercially available. Intravenous delivery is often disadvantageous in that it can be painful, uncomfortable, and inconvenient, resulting in poor patient compliance.

An alternative method which has been described for improving the bioavailabilty of itroconazole in pharmaceutical compositions for oral administration involves forming beads by incorporating the drug in hydrophilic polymers and applying the mixture on to a core. This approach is illustrated in U.S. Pat. No. 5,633,015 which is incorporated herein by reference in its entirety. Capsules of this type, for oral administration, comprising beads having a sugar core coated with a mixture of itraconazole and hydroxypropylmethylcellulose sealed with an outer coating layer are available commercially as Sporanox™ capsules (Janssen).

The bioavailability of itraconazole from the currently available oral capsules has been found to be variable, depending on whether dosing occurs before or after consumption of food, and is unpredictable from patient to patient. Moreover, due to the dissolution and bulk density characteristics of solid forms, acceptable dose size is problematic. Further, existing manufacturing methods involve complicated process steps and require the use of specialized manufacturing equipment.

The formation of solid dispersions to improve the solubility, and hence the dissolution characteristics, of itroconazole has been investigated. In International Journal of Pharmaceutics 187 (1999) 209-218, tablets containing solid dispersions of itraconazole with the pH dependent polymers polyvinylacetal diethylaminoacetate (AEA™) and aminoalkyl methacrylate copolymers (Eudragit™ E 100), prepared by spray-drying, were reported to show enhanced solubility of itraconazole compared to solid dispersions prepared by spray-drying with the pH independent hydrophilic polymers polyethylene glycol (PEG) 20,000, polyvinylpyrrolidine (PVP), polyoxyethylene-polyoxypropylene copolymers (Polaxamer 188) and hydroxypropylmethylcellulose (HPMC). The spray dried solid dispersions with the pH dependent polymers were reported to show enhanced dissolution profiles of itraconazole compared to those obtained from the marketed product.

Spray-drying process of the prior art, however, requires specialized equipment, and can be a costly way to formulate. Additionally, due to variabilities inherent in the spray-drying process, the dissolution, solubility and bioavailability characteristics of the resulting product may vary considerably.

Accordingly, there remains a need for the development of formulations and co-formulations comprising itraconazole, and methods for formulating and delivering formulations and co-formulations comprising itraconazole, and pharmaceutical compositions comprising itraconazole. In particular, there remains a need for formulations and methods for formulating itraconazole, to provide good solubility and/or dissolution characteristics, which formulation can be administered orally, resulting in good bioavailability, as well as having acceptable physical and chemical stability, as an alternative to the currently marketed products.

It is therefore desirable to improve itraconazole-containing formulations, co-formulations and compositions, and methods for preparing itraconazole containing formulations, co-formulations and compositions, such as for oral administration. It is particularly desirable to provide itraconazole containing co-formulations having comparable, or preferably enhanced, bioavailability compared to commercially available Sporanox™ capsules and which can be efficiently, readily and cost-effectively manufactured on an industrial scale.

### SUMMARY OF THE INVENTION

The present inventors have found that dissolution of itraconazole was not significantly enhanced by particle size reduction alone. The present inventors have further found that itraconazole can be formulated with oligomeric and/or polymeric excipients to give products which exhibit acceptable and/or improved solubility and/or dissolution characteristics and/or stability for pharmaceutical use. By means of the method and processes of the present invention, the compositions, especially the pharmaceutical compositions herein, may be provided using a relatively simple, efficient, reliable and cost-effective solid dispersion manufacturing technique.

According to one aspect, the invention comprises various formulations of itraconazole. Also provided are pharmaceutical compositions comprising itraconazole, methods for the preparation of itraconazole formulations and/or compositions, and the use of itraconazole formulations and/or compositions.

In another aspect of the invention, a formulation comprises a co-formulation of itroconazole and one or more oligomeric or polymeric excipients, the co-formulation prepared by a Gas Anti-Solvent (GAS) precipitation particle formation method. In embodiment of the present invention, the GAS precipitation method used to coformulate the itraconazole with another substance comprises a a Nektar™ SCF particle formation process, also known as the “SEDS™” (Solution Enhanced Dispersion by Supercritical fluids) process. In one embodiment of this process involves using the anti-solvent fluid simultaneously both to extract the vehicle from, and to disperse, the target solution/suspension.

In another aspect of the invention, a formulation comprises a co-formulation of itraconazole and one or more oligomeric or polymeric excipients, which co-formulation exhibits improved characteristics, which characteristics may comprise one or more of enhanced dissolution, solubility, stability, shelf life, bioavailability, or manufacturing ease or manufacturing cost-effectiveness.

In another aspect of the invention, a formulation comprises a co-formulation of itraconazole and one or more oligomeric or polymeric excipients, wherein the itraconazole is present in crystalline form.

In another aspect of the invention, a formulation comprises a co-formulation of itraconazole and one or more oligomeric or polymeric excipients, wherein the itraconazole is present in non-crystalline form.

In another aspect of the invention, a formulation comprises a co-formulation of itraconazole and polyvinylpyrrolidone.

In another aspect of the invention, a formulation comprises a co-formulation of itraconazole and hydroxypropylmethylcellulose.

In another aspect of the invention, a method of preparing a formulation comprises co-formulating itraconazole and one or more oligomeric or polymeric excipients using a GAS particle precipitation method which provides improved characteristics, which characteristics may comprise one or more of enhanced dissolution, solubility, good handling properties, chemical stability, physical stability, shelf life, bioavailability, manufacturing ease, and manufacturing cost-effectiveness.

In another aspect of the invention, a method of preparing a formulation comprises co-formulating crystalline itraconazole and one or more oligomeric or polymeric excipients.

In another aspect of the invention, a method of preparing a formulation comprises co-formulating non-crystalline itraconazole and one or more oligomeric or polymeric excipients.

In another aspect of the invention, a method of preparing a formulation comprises co-formulating itraconazole and polyvinylpyrrolidone using a GAS particle precipitation method which provides improved characteristics.

In another aspect of the invention, a method of preparing a formulation comprises co-formulating itraconazole and hydroxypropylmethylcellulose.

In another aspect of the invention, a method of preparing a formulation comprises co-formulating itraconazole and polyvinylpyrrolidone.

### BRIEF DESCRIPTION OF THE DRAWINGS

FIG. 1 is a schematic diagram of one embodiment of an apparatus for carrying out a particle precipitation process according to the present invention;

FIG. 2 is a schematic diagram of one of the components of the apparatus of FIG. 1;

FIG. 3 is a side elevational view, partially in cut-away, of parts of a fluid inlet assembly usable with the apparatus of FIG. 1;

FIG. 4 is a bottom plan view, of parts of the a fluid inlet assembly of FIG. 3;

FIGS. 5A and 5B are X-ray diffraction profiles of itraconazole prepared by co-formulating with PVP, using a GAS particle precipitation method. FIG. 5A was taken immediately after preparation, while FIG. 5B was taken after storage for six months at 40° C. and 75% relative humidity;

FIG. 6 is a graph of two dissolution profiles for a co-formulation made according to Example A (infra) labeled by the open triangle (Δ) and commercially available Sporanox™ capsules, labeled by the filled square (▪);

FIGS. 7A and 7B are SEM images of the starting itraconazole raw material (FIG. 7A) and the itraconazole/HPMC co-formulation product of Example B, infra (FIG. 7B). The images are at a magnification of 8000×;

FIG. 8 is a graph of dissolution profiles for the co-formulation of Example B (replicate analyses, labeled as triangles Δ and ▴) and commercially available Sporanox™ capsules (replicate analyses, labeled as squares ▪and □);

FIGS. 9A and 9B are X-ray diffraction profiles for a co-formulation of itraconazole with HPMC of Example B. FIG. 9A was taken immediately after preparation, while FIG. 9B was taken after storage for six months at 40° C. and 75% relative humidity;

FIG. 10 is a graph of dissolution profiles of itraconazole HPMC co-formulations using a GAS particle precipitation method produced at various ratios of itraconazole:HPMC;

FIG. 11 is a graph of dissolution profiles of itraconazole HPMC co-formulations using a GAS particle precipitation method produced at various operating pressures;

FIGS. 12A, 12B and 12C are SEM images of the itraconazole HPMC co-formulations using a GAS particle precipitation method produced at various operating pressures;

FIG. 13 is a graph of dissolution profiles of itraconazole HPMC co-formulations using a GAS particle precipitation method produced at various operating temperatures;

FIGS. 14A, 14B and 14C are SEM images of the itraconazole HPMC co-formulations using a GAS particle precipitation method produced at various operating temperatures;

FIG. 15 is a graph of dissolution profiles of itraconazole HPMC co-formulations using a GAS particle precipitation method produced at various process solution flow rates;

FIG. 16 is a graph of dissolution profiles of itraconazole HPMC co-formulations using a GAS particle precipitation method produced at various process solution concentrations;

FIGS. 17A and 17B are graphs of dissolution profiles of two itraconazole/HPMC co-formulations using a GAS particle precipitation method, at two different ratios of itraconazole:HPMC, and made in different vessel sizes, compared to a formulation of the prior art;

FIGS. 18A and 18B are SEM images of itraconazole produced using a co-formulation process of the present invention and wherein itraconazole is co-formulated with GMP compliant, 2910 substituted HPMC, as Pharmacoat 603-NF, manufactured by Shin Etsu Chemical. FIG. 18A shows a 60:40 ratio of itraconazole:HPMC, and FIG. 18B shows an 80:20 ratio of itraconazole:HPMC;

FIGS. 19A and 19B are SEM images of itraconazole produced using a using a GAS particle precipitation method co-formulation process of the present invention and wherein itraconazole is co-formulated with PVP (FIG. 19A) and HPMC (FIG. 19B); and

FIG. 20 is a graph of fraction absorbed v square root of time (adjusted for an in vivo absorption lag) for a co-formulation of the present invention comprising itraconazole and HPMC, made in accordance with Formulation Example B.

### DETAILED DESCRIPTION OF SPECIFIC EMBODIMENTS

Definitions

Before describing the present invention in detail, it is to be understood that the invention is not limited to the particularly exemplified apparatus, systems, methods, or processes disclosed herein, which may, of course, vary. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

It must be noted that, as used in this specification and the appended claims, the singular forms “a,” “an” and “the” include the plural unless the content clearly dictates otherwise

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

Amount of ingredients, materials or substances are listed as the ranges or levels of ingredients in the descriptions, which follow hereto.

“Therapeutically-effective amount” means that amount of active present in the composition that is needed to provide the desired level of drug in the subject to be treated to yield the expected physiological response.

“Drug” means any compound or composition which induces a desired pharmacologic and/or physiologic effect, when administered appropriately to the target organism (human or animal). Itraconazole is one example of a drug.

The term “vehicle” means a fluid which dissolves a solid or solids, to form a solution, or which forms a suspension of a solid or solids which do not dissolve or have a low solubility in the fluid. The vehicle can be composed of one or more fluids.

As used herein, a ‘co-formulation’ refers to two or more substances formulated at substantially the same time and/or formulated so that a particle comprising a co-formulation contains the two or more substances. For example, a co-formulation may comprise a solid dispersion of a first substance and a second substance, such as an intimate mixture of an active substance and an excipient. In one version, the intimate mixture may comprise an active agent, especially a pharmaceutically-active agent, such as itraconazole, dispersed in a “matrix” of a carrier material, especially an excipient, such as an oligomeric and/or polymeric excipient. The co-formulations of the present invention with an excipient may advantageously modify the solubility and/or dissolution characteristics of the active substance.

The term “crystalline” is intended to mean any solid which gives a wide angle x-ray diffraction pattern showing one or more of a set of peaks characteristic of the solid (the “diffraction pattern”) due to its three dimensional internal molecular structure or order, including pure compounds and mixtures which show such peaks.

“Non-crystalline” refers to any solid which does not give rise to one or more characteristic peaks in wide angle x-ray diffraction indicative of crystallinity as defined above. In the case of a single substance, this includes amorphous materials, which are disordered at the molecular level, and frozen thermotropic liquid crystals, which can be distinguished from amorphous materials because they exhibit birefringence under polarized light. In the case of a mixture, this includes molecular solid dispersions, which are comparable to liquid solutions in that there is a single phase which is disordered at the molecular level, and non-molecular solid dispersions, which have one or more distinct amorphous phases.

A Gas Anti-Solvent (GAS) precipitation method is meant to include, but is not limited to, a particle formation method as described in by Gallagher et al, ACS Symp. Ser., 406, p 334 (1989) or versions thereof such as are disclosed for instance in EP-0 322 687, WO-90/03782 and WO-97/31691. Without limiting the teachings of the foregoing references, a GAS process involves contacting a solution or suspension of the target substance(s) in a fluid vehicle (the “target solution/suspension”) with a compressed fluid (generally a supercritical or near-critical fluid) anti-solvent under conditions which allow the anti-solvent to extract the vehicle from the target solution/suspension and to cause particles of the target substance(s) to precipitate from it. The conditions are such that the fluid mixture formed between the anti-solvent and the extracted vehicle is still in a compressed (generally supercritical or near-critical) state. It is preferred that the anti-solvent fluid be a nonsolvent for the target substance(s) and be miscible with the fluid vehicle.

Itraconazole as used herein comprises the free base form, as well as pharmaceutically acceptable addition salts of itraconazole, or one of its stereoisomers, or a mixture of two, three of four stereoisomers. In preferred embodiments of the present invention, the itraconazole comprises the (±)-(2R*, 4S*) or (cis) forms of the free base. Moreover, other azole antifungal agents having similar chemical, physical, and physiological properties can be substituted for, or combined with itraconazole. Thus saperconazole, ketoconazole, posaconazole and oriconazole, to name a few, may be similarly employed in the formulations, co-formulations, pharmaceutical compositions methods of making and using, and applications of the present invention.

In one embodiment of the present invention, the process used to coformulate the itraconazole with another substance comprises a GAS precipitation method, and particularly a Nektar™ SCF particle formation process, also known as the “SEDS™” (Solution Enhanced Dispersion by Supercritical fluids) process. In one embodiment of this process involves using the anti-solvent fluid simultaneously both to extract the vehicle from, and to disperse, the target solution/suspension. In this context, ‘disperse’ refers generally to the transfer of kinetic energy from one fluid to another, usually implying the formation of droplets, or of other analogous fluid elements, of the fluid to which the kinetic energy is transferred. In one aspect of the invention, the SEDS™ process provides for the coformulation of an active (e.g. itraconazole) substance and an oligomeric or polymeric excipient, comprising an intimate single-phase mixture of the active substance and the excipient. The SEDS™ technique can thus provide better, and more consistent, control over the physicochemical properties of the product (particle size and size distribution, particle morphology, etc.) than has proved possible for co-formulations of the prior art.

In one particular embodiment, the target solution/suspension and the anti-solvent are contacted with one another by being co-introduced into a particle formation vessel using a fluid inlet which allows the mechanical energy (typically the shearing action) of the anti-solvent flow to facilitate intimate mixing and dispersion of the fluids at the point where they meet, as described for example in WO-95/01221 and corresponding U.S. Pat. No. 5,851,453, and/or WO-96/00610 and corresponding U.S. Pat. No. 6,063,138, and U.S. Published Patent Application Numbers 2002-0010982 and 2002-0073511, all of which are herein incorporated by reference in their entireties. The target solution/suspension and the anti-solvent may meet and enter the particle formation vessel at substantially the same point, for instance via separate passages of a multi-passage coaxial nozzle.

Alternatively or additionally, a process of the type described in WO-03/008082, and corresponding U.S. Published Patent Application Numbers 2003-0109421, 2003-0232020 and 2003-0223939, all of which are herein incorporated by reference in their entireties, may be utilized. In this process, the target solution/suspension and the anti-solvent enter the vessel at separate, although close, locations and the anti-solvent velocity as it enters the particle formation vessel is preferably sonic, near-sonic or supersonic. Generally speaking, in the method of the invention, a Mach number for the anti-solvent fluid on entering the particle formation vessel may be between 0.8 and 1.5, preferably between 0.9 and 1.3.

Reference to an anti-solvent fluid being in a compressed state means that, at the relevant operating temperatures, it is above its vapour pressure, preferably above atmospheric pressure, more preferably from 70 to 250 bar. The anti-solvent fluid is preferably a fluid which is a gas at atmospheric pressure and ambient temperature. The anti-solvent used is, in one embodiment, supercritical, near-critical or liquid CO₂, especially supercritical CO₂. The anti-solvent expands as it enters the particle formation vessel in an isenthalpic manner. Thus, an appropriate temperature for the anti-solvent upstream of the vessel may be derived from enthalpy charts for the anti-solvent.

In one embodiment of the present invention, ‘compressed’ means close to, at or more preferably above the critical pressure P_{c} for the fluid concerned. The anti-solvent is preferably a supercritical or near-critical fluid or may alternatively be a compressed liquid. A “supercritical fluid” is a fluid at or above its critical pressure (P_{c}) and its critical temperature (T_{c}) simultaneously. A ‘near-critical fluid’ is either (a) above its T_{c} but slightly below its P_{c} or (b) above its P_{c} but slightly below its T_{c} or (c) slightly below both its P_{c} and T_{c}.

The terms “compressed fluid”, “supercritical fluid” and “near-critical fluid” each may comprise a mixture of fluid types, so long as the overall mixture is in the compressed, supercritical or near-critical state respectively.

Suitable solvents for suspending/dissolving the target active or substance(s) comprise generally, hydroxylic, especially alcohol, solvents. Preferred are one or more of methanol, ethanol, isopropyl alcohol, acetone, tetrahydrofuran, ethyl acetate, dimethylformamide, dichloromethane (DCM), methanonitrile, dimethylacetamide and mixtures thereof. Most preferred is a mixture of dichloromethane and methanol.

The processing conditions are preferably chosen, as described in WO-03/008082 to produce particles of desired sizes and/or to reduce residual solvent levels. Process conditions which may be varied to achieve the desired result comprise vessel size, process temperature and pressure, supercritical fluid and process solution line orifice diameters (“aperture size”), process solution flow rate, supercritical fluid flow rate, and process solution concentration.

In the co-formulation with one or more oligomeric or polymeric excipients according to the invention, itraconazole may comprise a non-crystalline, such as amorphous, form. The amorphous form is more readily soluble but less stable than the crystalline form. It has been found that a co-formulation in accordance with the present invention is readily soluble, and chemically and physically stable. Thus, in one embodiment of the co-formulation and/or composition of the present invention, itraconazole comprises its non-crystalline form.

It has been found that crystalline itraconazole, which is more stable but less readily soluble than the non-crystalline form, may be co-formulated with one or more oligomeric or polymeric excipients to provide a stable co-formulation having at least parity, preferably improved solubility and/or dissolution rate characteristics compared to commercially available Sporanox™ capsules. Thus, in one embodiment of the co-formulation and/or composition of the present invention, itraconazole comprises its crystalline form. In such an embodiment, the crystalline form of itraconazole is preferably present in an amount of 1 to 100%. Preferably at least 99% or 95% or 80% or 70% or 60% or 50% or 40% or 30% or 20% or 10% of the itraconazole is in its crystalline form.

Itraconazole is present in a therapeutically effective amount in the formulation(s) of the present invention according to the target condition to which it is intended. In particular itraconazole is present in an amount which is effective as an antifungal agent. In powder form in accordance with the formulations, co-formulations, compositions and methods of making of the present invention, the itraconazole is present in an amount of from 10% to 95%, preferably from 30% to 90%, especially 50% to 85% w/w of the co-formulation and/or composition. In embodiments of the present invention comprising pharmaceutical compositions, the itraconazole may comprise 10% to 40% w/w, more preferably 20% to 30% w/w of the composition. In one embodiment of the present invention a co-formulation, in powder form, is blended with additional excipients, for example, microcrystalline cellulose sodium starch glycolate, or mixtures thereof.

The excipient in the co-formulation according to the invention may be any suitable excipient for the active substance, of whatever molecular weight, and suitably may be hydrophilic or hydrophobic. Preferably the excipient is non-toxic and pharmaceutically acceptable.

The excipient may comprise a single substance or a mixture of two or more, and may be monomeric, oligomeric or polymeric (typically either oligomeric or polymeric). It may be organic (including organometallic) or inorganic, hydrophilic or hydrophobic. It may be a carbohydrate, such as a mono, di or poly saccharide, cyclodextrin, or starch. It is typically a substance capable of protecting an active substance from external effects such as heat, light, moisture, oxygen or chemical contaminants, and/or of reducing incompatibilities between the active substance and another material with which it needs to be processed or stored, and/or of targetting, or altering the speed or timing of, the release of the active substance (for instance, for drug delivery systems), and/or of masking the flavour and/or odour of an active substance, when applied to the surface of the active substance. It is preferably non-toxic and pharmaceutically acceptable. In preferred embodiments of the formulations, co-formulations, compositions and methods of formulating and co-formulating of the present invention the excipient comprises a hydrophilic polymer, especially a hydrophilic polymer such as a hydroxypropyl methyl cellulose.

Examples of suitable excipients include celluloses and cellulose derivatives, such as alkyl (for example, methyl or ethyl) cellulose, hydroxyalkyl celluloses (such as hydroxypropylmethyl cellulose, hydroxypropylmethyl cellulose phthalate, hydroxyethyl cellulose, hydroxypropyl cellulose), carboxymethylcelluose, sodium carboxymethyl cellulose, microcrystalline cellulose, microfine cellulose) or mixtures thereof; traditional “natural” source materials, their derivatives and their synthetic analogues, such as acacia, tragacanth, alginates (for instance calcium alginate), alginic acid, starch, agar, carrageenan, xanthan gum, chitosan, gelatin, guar gum, pectin, amylase or lecithin; homo- and co-polymers of hydroxy acids such as lactic and glycolic acids; hydrated silicas, such as bentonite or magnesium aluminium silicate; polymeric surfactants, such as polyoxyethylene or polyoxypropylene, or polyalkylene oxides such as polyethylene oxides; phospholipids, such as DMPC (dimyristoyl phosphatidyl choline), DMPG (dimyristoyl phosphatidyl glycerol) or DSPC (distearyl phosphatidyl choline); carbohydrates, such as lactose, dextrans, cyclodextrins or cyclodextrin derivatives; dendrimeric polymers, such as those based on 3,5 hydroxy benzyl alcohol; poly(ε-caprolactones), DL-lactide-co-caprolactones and their derivatives; poly(orthoester)s and poly(orthoester)/poly(ethylene glycol) copolymers, including block copolymers, such as are described in U.S. Pat. No. 5,968,543 and U.S. Pat. No. 5,939,453, also derivatives of such polymers, also such polymers with incorporated esters of short chain a-hydroxy acids or glycolic-co-lactic acid copolymers; vinyl polymers (such as polyvinyl chloride, polyvinyl alcohols, polyvinyl acetates, polyvinyl pyrrolidones, cross-linked polyvinyl pyrrolidones or carboxy vinyl copolymers) or acrylates and their derivatives, such as the Eudragit™ polymers; or mixtures thereof.

In one embodiment of the composition of the present invention, the excipient comprises one or more cellulose or cellulose derivatives, such as methyl or ethyl cellulose, hydroxypropylmethyl cellulose, hydroxypropylmethyl cellulose phthalate, hydroxyethyl cellulose, hydroxypropyl cellulose, carboxymethylcelluose, sodium carboxymethyl cellulose, microcrystalline cellulose, microfine cellulose, or a mixture thereof. In one embodiment nof the present invention, the excipient comprises hydroxypropylmethylcellulose (HPMC). A preferred hydroxypropylmethylcellulose is marketed by Shin-Etsu Chemical and sold under the trademark Pharmacoat™. Particularly preferred is Pharmacoat™ 603, having about 29% hydroxyl groups and about 10% hydroxypropyl groups, and wherein a 2 wt. % aqueous solution at 20° C. has a viscosity of about 3 centipoise.

Additionally or alternatively, the excipient may comprise a polymer. In one particular version, the excipient comprises a vinyl polymer, such as polyvinylpyrrolidone. Other vinyl polymers, such as polyvinyl acetate/alcohol may also be suitable.

The excipient is present in an amount by weight sufficient, following formulation with itraconazole, to provide a stable formulation, or to provide dissolution and/or solubility and/or bioavailability characteristics at least equal to that of commercially available dry products. Preferably, the excipient is present in an amount to provide a both stable formulation, and to provide dissolution and/or solubility and/or bioavailability characteristics at least equal to, or better than, that of commercially available dry products. Generally, the oligomeric and/or polymeric excipient is present at a concentration in the range of from 1 to 99% w/w, preferably from 5% to 70%, more preferably from 10% to 50% w/w of the formulation.

In one embodiment, a co-formulation according to the invention comprises itraconazole and the excipient present in a weight ratio of 1:1. In another embodiment the itraconazole: excipient ratio is 40:60, or 60:40, or 80:20. For example, in one version, the co-formulation comprises itraconazole and hydroxypropylmethylcellulose, in a weight ratio of 1:1. In one version, the itraconazole of this version is present in its crystalline form.

In one version, the co-formulation according to the invention contains no or only minor amounts (for example, less than 5% w/w, preferably less than 2% w/w) of additional ingredients, that is it consists essentially of itraconazole and the oligomeric and/or polymeric excipient or excipients. In one version, the co-formulation according to the invention contains no stabilizers, especially no surfactants.

In the formulations, co-formulations and compositions according to the invention, the itraconazole is physically stable for at least one month, preferably three months, more preferably at least six months, and most preferably for at least one year after its preparation. By ‘stable’ is meant that over the specified time period, there is no significant change in the X-ray diffraction (XRD) pattern of the active substance and/or, where measurable, in its differential scanning calorimetry (DSC) profile. The formulations, co-formulations and compositions according to the invention further exhibit good chemical stability, i.e. the resistance to the formation of impurities and/or related substances caused by chemical degradation. Chemical stability may be determined by any suitable method known to the art, for example, by HPLC methods.

Stability may suitably be assessed by storing the formulation according to the invention at ambient temperature (e.g., from 18 to 25° C., or from 20 to 23° C., such as about 22° C., or at the accepted industrial standard temperature of 25° C.), and at up to 20% or 30% or 40% or 60% or even 75% relative humidity (RH). Higher storage temperatures and/or humidities (such as storage at 40° C. and 75% RH) may be used, in conventional manner, to mimic longer term storage periods, as may conventional thermal cycling procedures such as freeze/thaw cycling.

The degree of crystallinity of the formulation may be assessed by conventional techniques, for example using X-ray diffraction (XRD) techniques, particularly high resolution X-ray powder diffraction (XRPD) using a synchrotron radiation source. Levels of amorphous phase may also be assessed by reference to its moisture uptake at any given temperature and humidity. XRD or XRPD profiles of crystalline substances exhibit characteristics peaks. Such characteristic peaks are absent in amorphous materials, where the diffraction pattern reveals only low-level background noise. Crystalline materials thus exhibit reduced diffraction line broadening and/or a higher signal to noise ratio than non-crystalline materials.

Bioavailability may be assessed, according to standard procedures, with reference to the release profile of the active substance, with time, into the patient's bloodstream. It may be measured for example as either the maximum plasma concentration of active achieved following administration (Cₘₐₓ), or as the area under the plasma concentration curve (AUC) integrated from time zero (the point of administration) to a suitable endpoint or to infinity.

A co-formulation according to the invention may be prepared by co-precipitating the two materials from a common solvent or solvent mixture using a compressed (typically supercritical or near-critical) fluid anti-solvent as in the GAS (Gas Anti-Solvent) precipitation method as described herein. Preferably, a co-formulation, may be prepared by coprecipitating itraconazole and the excipient from a common solvent or solvent mixture using the Nektar™ SCF particle formation process as described herein.

Using a Nektar™ SCF process, the target solution/suspension contains the active substance and the excipient in a common fluid vehicle (which may itself comprise a mixture of two or more fluids, either pre-mixed or mixed in situ at or immediately before the point of anti-solvent contact). In one embodiment of the present invention, the Nektar™ SCF process is most suitably of the type described in WO-02/38127, and its corresponding United States Published Patent Application Number 2002-0114844, the entire contents of which are herein incorporated by reference. These references disclose a GAS particle precipitation process in which the active substance and the excipient are coprecipitated from a common solvent system.

In another embodiment of the present invention, the Nektar™ SCF process may be of the type described in WO-03/008082, the entire contents of which are herein incorporated by reference, and/or in UK patent application No. 0300338.1 and/or GB Publication 2,398,241 to Kordikowski et al. In such processes, the target solution/suspension and the anti-solvent enter the vessel at separate, although close, locations and the anti-solvent velocity as it enters the particle formation vessel is ideally near-sonic, sonic or supersonic. Thus, a Mach number for the anti-solvent fluid on entering the particle formation vessel may be between 0.8 and 1.5, preferably between 0.9 and 1.3.

Co-formulations according to the invention when prepared by a GAS precipitation method, in particular a Nektar™ SCF particle formation process comprise discrete particles which are more free-flowing than corresponding co-formulations prepared by other solid dispersion processes, leading to improvements in handling and processability. Moreover, amorphous drug co-formulations prepared in this manner are more stable.

The Nektar™ SCF process is a process for forming particles of one or more “target” substances. It is a GAS process and so involves contacting a solution or suspension of the target substance(s) in a fluid vehicle (the “target solution/suspension”) with a compressed fluid (generally a supercritical or near-critical fluid) anti-solvent under conditions which allow the anti-solvent to extract the vehicle from the target solution/suspension and to cause particles of the target substance(s) to precipitate from it. The conditions are such that the fluid mixture formed between the anti-solvent and the extracted vehicle is still in a compressed (generally supercritical or near-critical) state. The anti-solvent fluid should be a nonsolvent for the target substance(s) and be miscible with the fluid vehicle.

Examples of Nektar™ SCF processes are described in WO-95/01221, WO-96/00610, WO-98/36825, WO-99/44733, WO-99/59710, WO-01/03821, WO-01/15664, WO-02/38127 and WO-03/008082. Other suitable processes are described in WO-99/52507, WO-99/52550, WO-00/30612, WO-00/30613, WO-00/67892 and WO-02/058674. All of these documents are incorporated herein by reference in their entireties.

Carrying out a Nektar™ SCF process specifically involves using the anti-solvent fluid simultaneously both to extract the vehicle from, and to disperse, the target solution/suspension. In other words, the fluids are contacted with one another in such a manner that the mechanical (kinetic) energy of the anti-solvent can act to disperse the target solution/suspension at the same time as it extracts the vehicle. “Disperse” in this context refers generally to the transfer of kinetic energy from one fluid to another, usually implying the formation of droplets, or of other analogous fluid elements, of the fluid to which the kinetic energy is transferred.

As used in the methods described herein, references to an anti-solvent fluid being in a compressed state mean that, at the relevant operating temperature, it is above its vapour pressure, preferably above atmospheric pressure, more preferably from 70 to 250 bar. The anti-solvent fluid is preferably a fluid which is a gas at atmospheric pressure and ambient temperature. In other words, it should have a vapour pressure above 1 bar at ambient temperature (e.g., at 18 to 25° C., such as at 22° C.).

More preferably “compressed” means close to, at or yet more preferably above the critical pressure P_{c} for the fluid concerned. The anti-solvent is preferably a supercritical or near-critical fluid, although it may alternatively be a compressed liquid such as for instance liquid CO₂. In practice, the pressure is likely to be in the range for example (0.7-3.0) P_{c}, preferably (0.7-1.7) P_{c}, for a compressed liquid anti-solvent such as liquid CO₂.

As used herein, the term “supercritical fluid” means a fluid at or above its critical pressure (P_{c}) and critical temperature (T_{c}) simultaneously. In practice, the pressure of the fluid is likely to be in the range (1.01-9.0) P_{c}, preferably (1.01-7.0) P_{c}, and its temperature in the range (1.01-4.0) T_{c} (measured in Kelvin). However, some fluids (e.g., helium and neon) have particularly low critical pressures and temperatures, and may need to be used under operating conditions well in excess of (such as up to 200 times) those critical values.

“Near-Critical fluid” refers to a fluid which is either (a) above its T_{c} but slightly below its P_{c} or (b) above its P_{c} but slightly below its T_{c} or (c) slightly below both its P_{c} and T_{c}. The term “near-critical fluid” thus encompasses both high pressure liquids, which are fluids at or above their critical pressure but below (although preferably close to) their critical temperature, and dense vapours, which are fluids at or above their critical temperature but below (although preferably close to) their critical pressure.

By “sonic velocity” and “supersonic velocity” is meant respectively that the velocity of the anti-solvent fluid as it enters the vessel is the same as or greater than the velocity of sound in that fluid at that point. By “near-sonic velocity” is meant that the anti-solvent velocity on entry into the vessel is slightly lower than, but close to, the velocity of sound in that fluid at that point—for instance its “Mach number” M (the ratio of its actual speed to the speed of sound) is greater than 0.8, preferably greater than 0.9 or 0.95. Generally speaking, in the method of the invention, the Mach number for the anti-solvent fluid on entering the particle formation vessel may be between 0.8 and 1.5, preferably between 0.9 and 1.3.

In one embodiment, the method of the present invention comprises a method for forming a substance, or co-forming two or more substances, in particulate form, the method comprising introducing into a particle formation vessel (a) a solution or suspension of the target substance in a fluid vehicle (the “target solution/suspension”) and (b) a compressed fluid anti-solvent for the substance, and allowing the anti-solvent fluid to extract the vehicle from the target solution/suspension so as to form particles of the target substance, wherein (i) the pressure in the particle formation vessel is P₁ which is preferably greater than the critical pressure P_{c} of the anti-solvent, (ii) the anti-solvent is introduced through a restricted inlet so as to have a back pressure of P₂, where P₂ is greater than P₁, (iii) the temperature in the particle formation vessel is T₁ which is preferably greater than the critical temperature T_{c} of the anti-solvent, (iv) the anti-solvent is introduced into the vessel at a temperature T₂, where T₂ is greater than T₁, (v) T₁ and T₂ are such that Joule-Thomson cooling of the anti-solvent as it enters the vessel does not reduce the anti-solvent temperature to below that required of it at the point of particle formation (and are preferably such that the anti-solvent temperature does not fall below T_{c} within the vessel) and (vi) P₁, P₂, T₁ and T₂ are such that the anti-solvent fluid has a sonic, near-sonic or supersonic velocity as it enters the particle formation vessel.

Although not wishing to be bound by theory, it is believed that in the method of the invention, a so-called “Mach disk” is generated in the anti-solvent flow downstream of the second fluid inlet means. In this region the fluid velocity will change abruptly to sub-sonic thus generating shock waves in the fluids present (in effect a continuous, low volume, supersonic boom). These shock waves are thought to aid mixing and dispersion of the target solution/suspension with the anti-solvent. Moreover they will propagate in the direction of the anti-solvent flow, rather than in a counter-current sense.

The arrangement of the first and second inlet means will preferably be such that the Mach disk is generated upstream (in the direction of anti-solvent flow) of the point of entry of the target solution/suspension into the particle formation vessel. It should occur in line with the longitudinal axis of the second inlet means, i.e., in line with the direction of anti-solvent flow.

The near-sonic, sonic or supersonic anti-solvent velocity is ideally achieved, in the method of the present invention, by the use of appropriate anti-solvent flow rates, back pressures and/or operating temperatures, and preferably without the aid of mechanical, electrical and/or magnetic input such as for example from impellers, impinging surfaces especially within the anti-solvent introducing means, electrical transducers and the like. Introducing the anti-solvent via a convergent nozzle, ideally as a single fluid stream, may also help in the achievement of appropriate fluid velocities.

The use of near-sonic, sonic or supersonic anti-solvent velocities can allow achievement of smaller particle sizes and narrower size distributions in GAS-based particle formation processes. In particular it can allow the formation of small micro- or even nano-particles, for instance of volume mean diameter less than 5 microns, preferably less than 2 microns, more preferably less than 1 micron. Such particulate products preferably have narrow size distributions, such as with a standard deviation of 2.5 or less, more preferably 2.0 or less, most preferably 1.9 or even 1.8 or less.

The use of near-sonic, sonic or supersonic anti-solvent velocities also appears to lead to more efficient vehicle extraction, thus potentially yielding particles with lower residual solvent levels, less agglomeration and generally improved handling properties.

Preferably the two fluids meet immediately downstream of the point of anti-solvent entry. “Immediately” in this context implies a sufficiently small time interval (between the anti-solvent entering the particle formation vessel and its contact with the target solution/suspension) as preferably still to allow transfer of mechanical energy from the anti-solvent to the solution/suspension so as to achieve dispersion. Nevertheless, there is still preferably a short interval of time between anti-solvent entry and fluid contact so as to eliminate, or substantially eliminate or at least reduce, the risk of apparatus blockage due to particle formation at the point of anti-solvent entry. The timing of the fluid contact will depend on the natures of the fluids, the target substance and the desired end product, as well as on the size and geometry of the particle formation vessel and the apparatus used to introduce the fluids and on the fluid flow rates. The contact may occur within 0.5 to 10 seconds, more preferably within 1 to 7 seconds, most preferably within 1.2 to 6 seconds, such as within 1.4 to 5.5 seconds, of the anti-solvent entering the particle formation vessel.

At the point where the target solution/suspension and the anti-solvent meet, the angle between their axes of flow may be from 0 degrees (i.e., the two fluids are flowing in parallel directions) to 180 degrees (i.e., oppositely-directed flows). However, they preferably meet at a point where they are flowing in approximately perpendicular directions, i.e., the angle between their axes of flow is from about 70 to 110 degrees, more preferably from about 80 to 100 degrees, such as about 90 degrees.

When carrying out the present invention, the particle formation vessel temperature and pressure are ideally controlled so as to allow particle formation to occur at or substantially at the point where the target solution/suspension meets the anti-solvent fluid. The conditions in the vessel must generally be such that the anti-solvent fluid, and the solution which is formed when it extracts the vehicle, both remain in the compressed (preferably supercritical or near-critical, more preferably supercritical) form whilst in the vessel. For the supercritical, near-critical or compressed solution, this means that at least one of its constituent fluids (usually the anti-solvent fluid, which in general will be the major constituent of the mixture) should be in a compressed state at the time of particle formation. There should at that time be a single-phase mixture of the vehicle and the anti-solvent fluid, otherwise the particulate product might be distributed between two or more fluid phases, in some of which it might be able to redissolve. This is why the anti-solvent fluid needs to be miscible or substantially miscible with the vehicle.

The flow rate of the anti-solvent fluid relative to that of the target solution/suspension, and its pressure and temperature, should be sufficient to allow it to accommodate the vehicle, so that it can extract the vehicle and hence cause particle formation. The anti-solvent flow rate will generally be higher than that of the target solution/suspension—typically, the ratio of the target solution/suspension flow rate to the anti-solvent flow rate (both measured at or immediately prior to the two fluids coming into contact with one another) will be 0.001 or greater, preferably from 0.01 to 0.2, more preferably from about 0.03 to 0.1. The anti-solvent flow rate will also generally be chosen to ensure an excess of the anti-solvent over the vehicle when the fluids come into contact, to minimize the risk of the vehicle re-dissolving and/or agglomerating the particles formed.

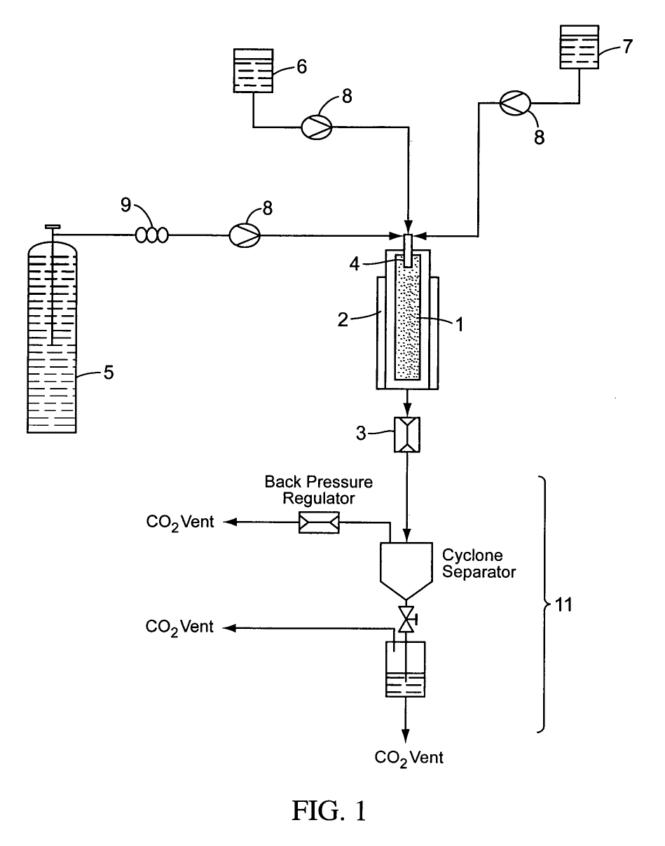

FIG. 1 shows on embodiment of an apparatus suitable for carrying out methods in accordance with the present invention. Reference numeral 1 denotes a particle formation vessel, within which the temperature and pressure can be controlled by means of a heating jacket 2 and back a pressure regulator 3. The vessel 1 contains a particle collection device (not shown) such as a filter, filter basket or filter bag. A fluid inlet assembly 4 allows introduction of a compressed (typically supercritical or near-critical) fluid anti-solvent from source 5 and one or more target solutions/suspensions from sources such as 6 and 7. The elements labeled 8 are pumps, and 9 is a cooler. A recycling system 11 allows solvent recovery.

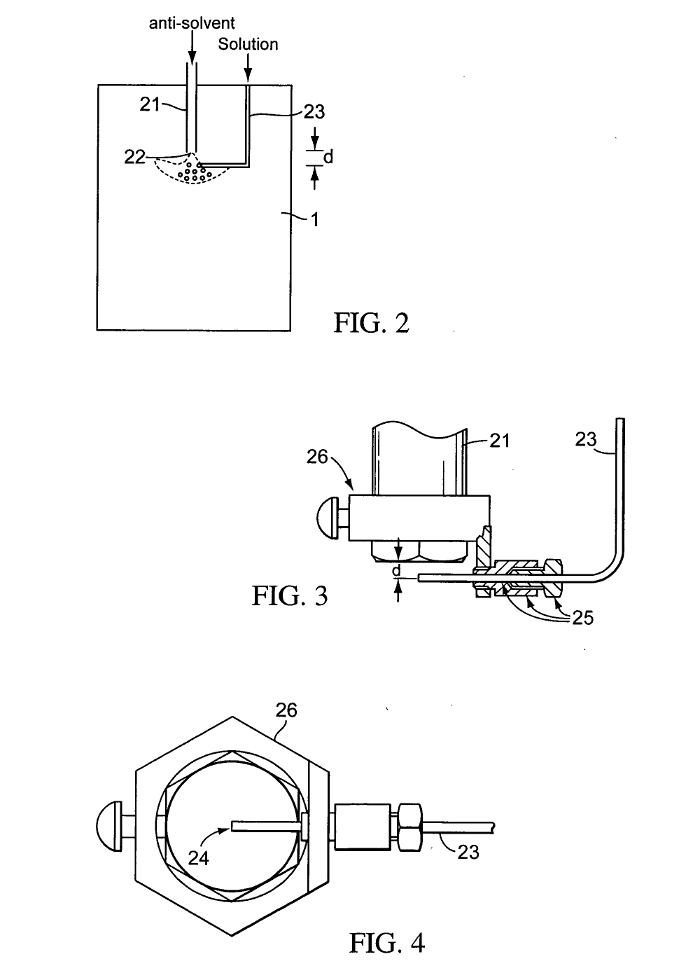

The fluid inlet assembly 4 may for example take the form shown in FIGS. 2-4. FIG. 3 shows the assembly schematically, in use with the particle formation vessel 1 of the FIG. 1 apparatus. Nozzle 21 is for introduction of the anti-solvent fluid. It is illustrated with only a single passage of circular cross section, with a circular outlet 22. However, nozzle 21 may alternatively comprise, a multi-component nozzle, with anti-solvent introduced through one or more of its passages and the remaining passages either closed off or else used to introduce additional reagents. (For example, a multi-passage nozzle of the type described in WO-95/01221 and/or corresponding U.S. Pat. No. 5,851,453 or WO-96/00610 may be used). Such nozzles have two or more concentric (coaxial) passages, the outlets of which are typically separated by a short distance to allow a small degree of internal mixing to take place between fluids introduced through the respective passages before they exit the nozzle. The anti-solvent could for instance be introduced through the inner passage of such a nozzle, traversing a small “mixing” zone as it exits that inner passage and then passing through the main nozzle outlet into the particle formation vessel).

Preferably, the opening at the outlet end (tip) of the nozzle 21 will have a diameter in the range of 0.05 to 2 mm, more preferably between 0.1 and 0.3 mm, typically about 0.2 mm. The outlet end of the nozzle 21 may be tapered depending upon the desired velocity of the fluids introduced through the nozzle; an increase in the angle may be used, for instance, to increase the velocity of the supercritical fluid introduced through the nozzle and hence to increase the amount of physical contact between the supercritical fluid and the vehicle.

Inlet tube 23 provides for the introduction of the target solution/suspension, and is so shaped and located that the direction of flow of the solution/suspension at its outlet 24 (see also FIG. 4) will be perpendicular to that of the anti-solvent exiting nozzle 21. Again the tube is preferably of circular cross section. The letter “d” refers to the distance between the outlets of nozzle 21 and tube 23, which, in some embodiments, may be adjustable to provide different particle characteristics.

FIG. 3 illustrates one aspect of tube 23 may be mounted, by means of the supporting and locking pieces 25, on a collar 26 which is itself mounted around the lower portion of the nozzle 21. The arrangement is such as to allow adjustment of the distance “d” between the outlets of nozzle 21 and tube 23. It can be seen that the outlet of tube 23 is positioned on the central longitudinal axis of the nozzle 21.

The co-formulations of the present invention may be further formulated into a pharmaceutical composition. A pharmaceutical composition according to the invention may suitably take any delivery form conventional in the art, particularly for oral administration. The composition may take the form of a solid composition such as a powder, granulate or tablet, for example, or a liquid form such as a solution or suspension (including more viscous forms such as pastes and gels) suitable for oral delivery. Also there are provided methods of treatment using a pharmaceutical composition according to the present invention. The invention thus further comprises methods of inhibiting a fungal infection in a patient by administering an effective amount of a pharmaceutical composition according to the present invention.

Pharmaceutical compositions according to the invention comprise an itraconazole containing co-formulation according to any of the above aspects together with a pharmaceutically acceptable carrier. For use as an anti-fungal agent, it will be appreciated that the pharmaceutical composition will comprise an anti-fungal effective amount of itraconazole in accordance with the invention as set forth above.

Pharmaceutical compositions according to the invention may comprise additional active substances and/or excipients, which may or may not be included along with the itraconazole and the excipient as part of the co-formulation of the invention. Suitably, pharmaceutical compositions according to the invention may include other additives such as those typically used in pharmaceutical dosage formulations, for instance flavourings and sweeteners, colours, bulking agents, tablet lubricants and disintegrating agents.

In one embodiment of the present invention, a pharmaceutical composition comprises an itraconazole formulation or co-formulation of itraconazole and excipient as described in any formulation, co-formulation, composition and method herein, together with additional excipients. In one embodiment, the additional excipients are blended with the itraconazole co-formulation, in powder form, and roller compacted, then filled into capsules. In a preferred embodiment, the pharmaceutical composition comprises a powder co-formulation of itraconazole, especially crystalline itraconazole, with an oligomeric or polymeric excipient, made by a GAS particle precipitation process. The powder co-formulation is then blended with microcrystalline cellulose and sodium starch glycolate, roller compacted, and filled into capsules. The blending step may comprise blending in a “V” blender. In another preferred embodiment, a pharmaceutical composition comprises a powder co-formulation of crystalline itraconazole with HPMC, made by the Nektar™ SCF GAS particle precipitation process. The powder co-formulation is then blended with microcrystalline cellulose and sodium starch glycolate, roller compacted, and filled into capsules. In one preferred embodiment, a composition comprising itraconazole has a bulk density appropriate to enable filling a single dose, especially a therapeutic dose, in a size 0 capsule.

Also provided are methods of treatment using a pharmaceutical composition according to the above aspect. The invention provides a method of treating a fungal infection in a patient in need of such treatment by administering an anti-fungal effective amount of a pharmaceutical composition as defined above. In a preferred method of treatment, the pharmaceutical composition is administered orally.

While the method of co-formulating has been described in terms of the SCF process, other processes may be used provided the desired stability and dissolution/solubility characteristics of the co-formulated product are attained. For example, spray drying, freeze drying, spray-freeze drying, fluid bed agglomeration may be employed, as long as the desired characteristics are attained. Thus, when using the crystalline form of the itraconazole, it is preferred that the dissolution and solubility characteristics of at least 91% release after 45 minutes is attained, while maintaining the itraconazole's stability. Similarly, when using the non-crystalline form of the itraconazole, it is preferred that the physical and/or chemical stability characteristic of remaining stable (as measured by XRD or DSC) for at least three months is attained, while maintaining the itraconazole's dissolution and solubility characteristics.

In preferred embodiments of the present invention, the co-formulations comprising a plurality of particles, e.g. a powder, have a bulk density of at least about 0.09 g/ml, or at least about 0.10 g/ml, preferably at least about 0.11 g/ml, or a tap density of at least about 0.12 g/ml, or at least about 0.13 g/ml, preferably at least about 0.14 g/ml. In one embodiment of the formulations, co-formulations, compositions and methods of making described herein, a bulk density is at least about 0.14 or 0.15 g/ml, and/or a tap density is at least about 0.18 or 0.19 g/ml. For embodiments comprising particulate formulations or co-formulations, a preferred mean particle size is between about 3 and 8 microns, more preferably between about 4 and 6 microns. Additionally, it is preferred that the formulations, co-formulations, and pharmaceutical compositions of the present invention exhibit a release percentage of at least about 91%, preferably at least about 93%, more preferably at least about 94%, and most preferably at least about 95%, after 45 minutes. The formulations, co-formulations or compositions of the present invention are preferably free (or easy) flowing, having discrete particles which are relatively non-cohesive compared to formulations of the prior art. In one embodiment of the present invention, there is provided a pharmaceutically-acceptable formulation of itraconazole, which is in a simplified formulation, and which is readily manufacturable, and physically and pharmaceutically stable during throughout manufacture, distribution, shipping and consumer shelf-life.

In a preferred embodiment of a composition and/or pharmaceutical composition according to the present invention, itraconazole is co-formulated with HPMC in a ratio of itraconazole:HPMC of 50:50 or 60:40 or 80:20. In a more preferred composition and/or pharmaceutical composition according to the present invention, itraconazole is co-formulated, using the Nektar™ SCF particle formation process, with HPMC in a ratio of itraconazole:HPMC of 50:50 or 60:40 or 80:20. In a most preferred embodiment of a composition and/or pharmaceutical composition according to the present invention, crystalline itraconazole is co-formulated, using the Nektar™ SCF particle formation process, with HPMC in a ratio of itraconazole:HPMC of 50:50 or 60:40 or 80:20.

In a preferred embodiment of a composition and/or pharmaceutical composition according to the present invention, itraconazole is co-formulated with PVP in a ratio of itraconazole:PVP of 50:50 or 60:40 or 80:20. In a more preferred composition and/or pharmaceutical composition according to the present invention, itraconazole is co-formulated, using the Nektar™ SCF particle formation process, with PVP in a ratio of itraconazole:PVP of 50:50 or 60:40 or 80:20. In a most preferred embodiment of a composition and/or pharmaceutical composition according to the present invention, crystalline itraconazole is co-formulated, using the Nektar™ SCF particle formation process, with PVP in a ratio of itraconazole:PVP of 50:50 or 60:40 or 80:20.

The following formulation and process examples serve to illustrate the advantages of the various embodiments of the formulations and methods of making and methods of using of the present invention, which may be further illustrated by reference to the following non-limiting examples and the drawing figures.

### EXPERIMENTAL DETAILS

### Particle Formation Process Examples

The following Examples illustrate the preparation of co-formulations of itraconazole and various excipients in accordance with the present invention. The excipient materials were: hydroxypropylmethylcellulose (6 cps solution viscosity) from Sigma-Aldrich and a low molecular weight (about 3500 g/mole) polyvinylpyrrolidone, having a low viscosity (K12 viscosity value), from Acros Organics.

Co-formulations comprising itraconazole and excipient were prepared using a supercritical fluid particle precipitation process, comprising essentially the Nektar™ SCF particle precipitation process of the type described in FIGS. 1-4, and in WO 03/008082. In this method, the nozzles are arranged such that the direction of flow of the itraconazole containing solution is perpendicular to the flow of the anti-solvent. The anti-solvent is introduced at a near-sonic, sonic or supersonic velocity. Supercritical carbon dioxide—the anti-solvent—was introduced through at a flow rate of 12-12.5 kg/hr and a solution of the drug (itraconazole) and polymer (2.5% w/v) was introduced at a flow rate of 1 ml/min. The pressure in the particle formation vessel was 80 bar and the temperature was 35° C. The solvent used is described in the Example.

### Formulation Example A—Preparation of Co-formulations Comprising Itraconazole and Polyvinyl Pyrrolidone

Itraconazole was co-formulated with polyvinyl pyrrolidone using the Nektar™ SCF particle precipitation process as described above in a drug:polymer ratio of 1:1. Tetrahydrofuran was used as the drug/polymer solvent. The resulting product was in the form of a dispersed particulate powder which was non-cohesive and easy-flowing with good handling properties.

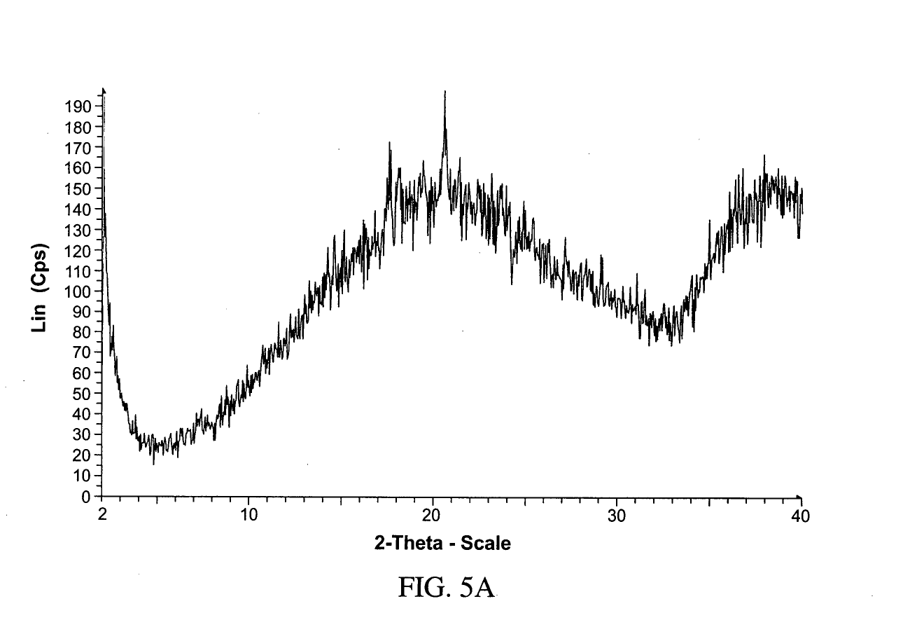

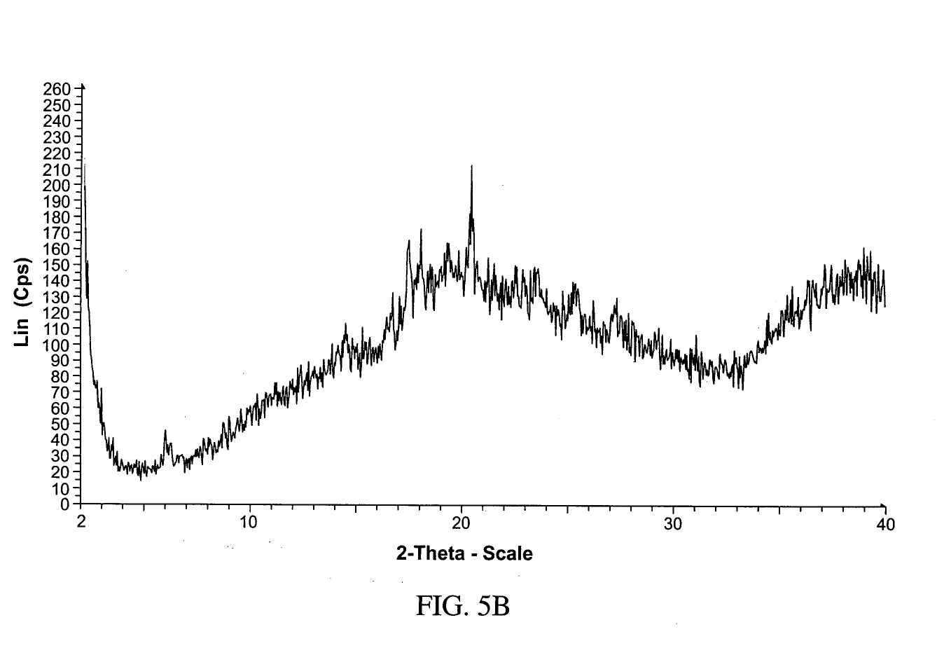

XRD showed that the product was amorphous (See FIG. 5A, wherein the amorphous nature is illustrated by the absence of significant characteristic peaks in the diffraction pattern). Samples were stored (in the form of the as-prepared powder) at 40° C. and 75% relative humidity in capped vials with smaller samples being removed at intervals and their crystallinity assessed using XRD. All samples were found to be stable (that is, remaining amorphous) after storage for six months under these conditions (See FIG. 5B, wherein the diffraction pattern is substantially unchanged from that of FIG. 5A).

The dissolution profile of the product of Example A was carried out using an eight bath Copley dissolution kit with attached UV Spectrophotometer. The apparatus comprised an Erweka DT800 low head dissolution tester; an Ismatec IPC 8-channel peristaltic pump; a Perkin Elmer Lambda 25 Spectrophotometer with cell changer and an Erweka fraction collector. The system is PC-controlled, via a Dissobox control unit.

The conditions used were itraconazole/PVP: 285 nm, 37±1° C., stirrer speed 75 rpm, 900 ml (1% sodium dodecyl sulphate in BP Artificial Gastric Fluid with no pepsin): tested as a 50:50 blend with lactose.

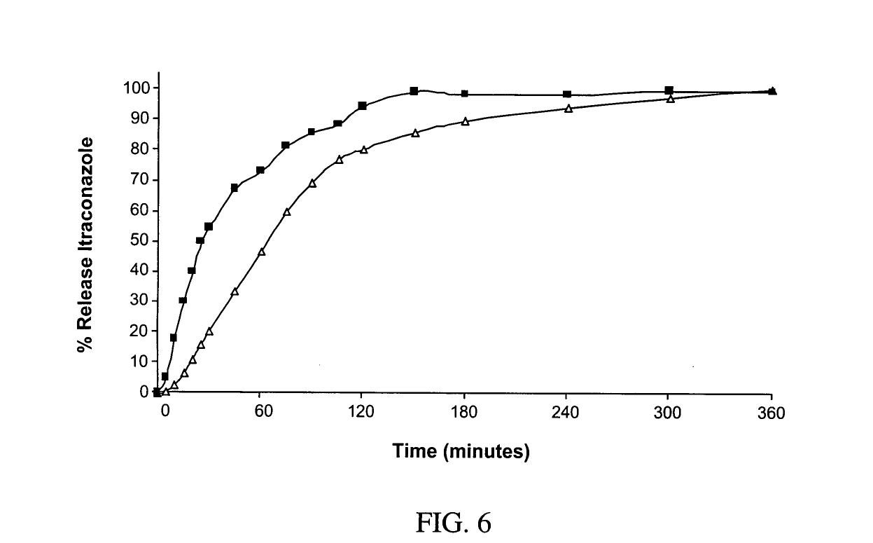

The release profile characteristics of the product of Example A (labeled as open triangles) were compared with the dissolution profile measured for the commercially available Sporanox™ capsules product (labeled as filled squares). The results are presented in FIG. 6. From this figure it can be seen that the dissolution profile of the co-formulation blended with lactose is comparable to that of the commercially available product.

### Formulation Example B—Preparation of Co-formulations Comprising Itraconazole and Hydroxypropylmethylcellulose

Itraconazole was co-formulated with hydroxypropylmethylcellulose using the Nektar™ SCF particle precipitation process as described above in a drug:polymer ratio of 1:1. Dichloromethane:methanol in a 1:1 ratio was used as the drug/polymer solvent. The product was in the form of a finely dispersed particulate powder which was non-cohesive and easy-flowing with good handling properties.

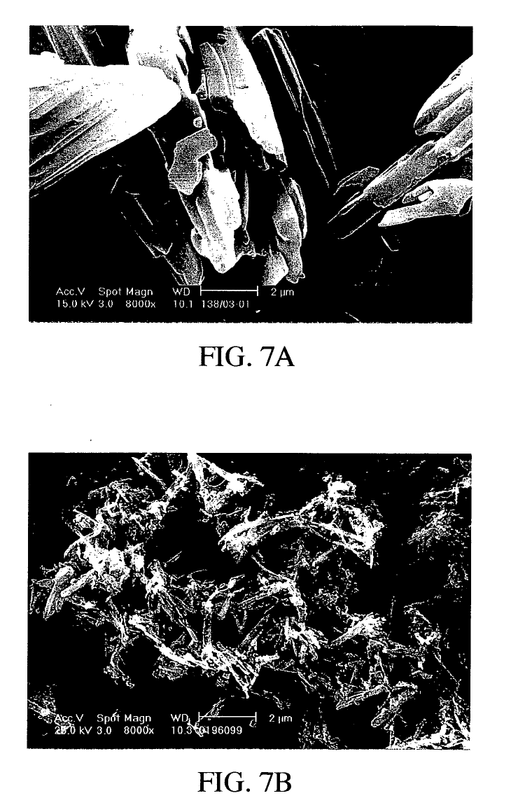

SEM studies confirm that in the co-formulation the itraconazole is present in crystalline form and the polymer in amorphous form. FIG. 7A shows SEM images of the starting itraconazole raw material and FIG. 7B shows the itraconazole/HPMC co-formulation product of the example (at 8000× magnification). It can clearly be seen that in the co-formulated product, the particle size of the itraconazole is much smaller than in the starting material. Smaller particles are not only easier to process and handle than larger particles but also would be expected to show improved solubility and hence more advantageous dissolution characteristics due to increased surface area. In addition, the small itraconazole crystals are encrusted with the amorphous polymer (i.e. the polymer is deposited on the itraconazole crystals). Such a configuration would be expected to impart to the co-formulation improved solubility and hence improved, and more advantageous, dissolution characteristics by several possible modes of action, including improved wetting. This therefore represents a significant advantage for the co-formulation of itraconazole with HPMC according to the invention.

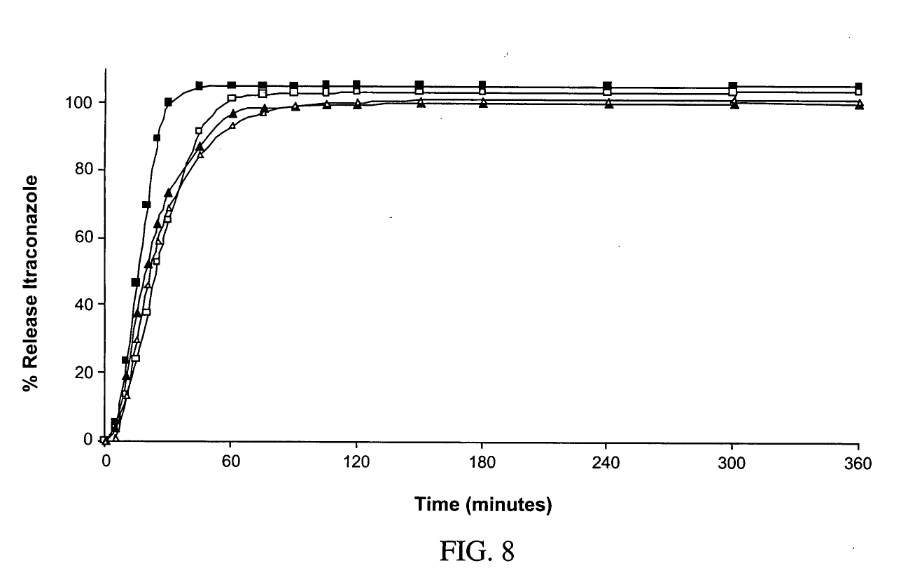

The dissolution profile of the product of Example B was obtained using the test procedure described for Example A, above. The conditions used were itraconazole/HPMC: 285 nm, 37±1° C., stirrer speed 100 rpm, 900 ml (1% sodium dodecyl sulphate in BP Artificial Gastric Fluid. The results are presented in FIG. 8. The curves marked by the light and dark triangles represent two different trials of the co-formulation of the present invention, while the curves marked by the light and dark squares represent two trials of the commercially-available Sporanox™ capsules product. It can be seen that the dissolution profile of the co-formulation with HPMC is comparable to that of the commercially available product.

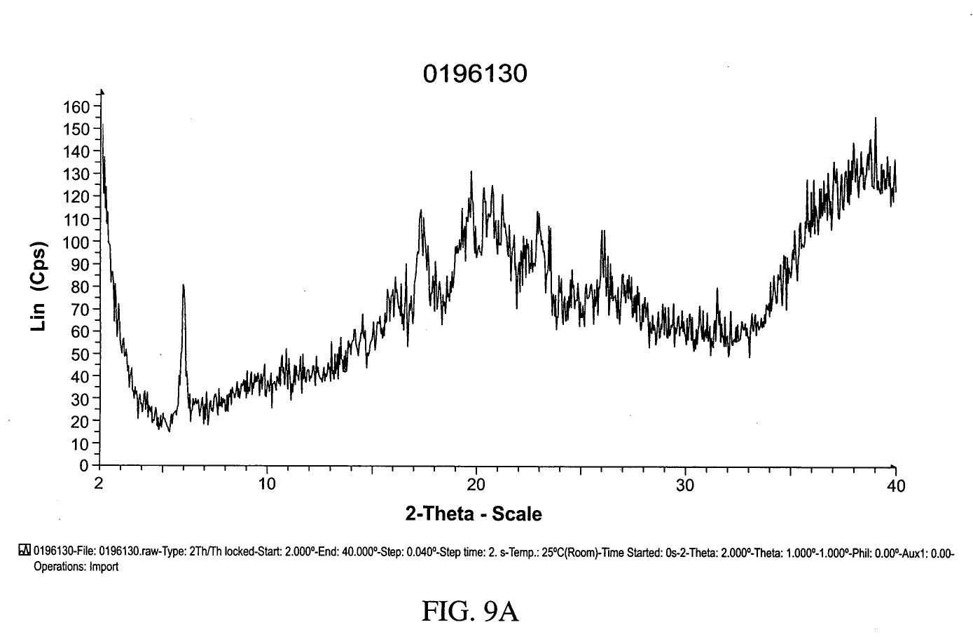

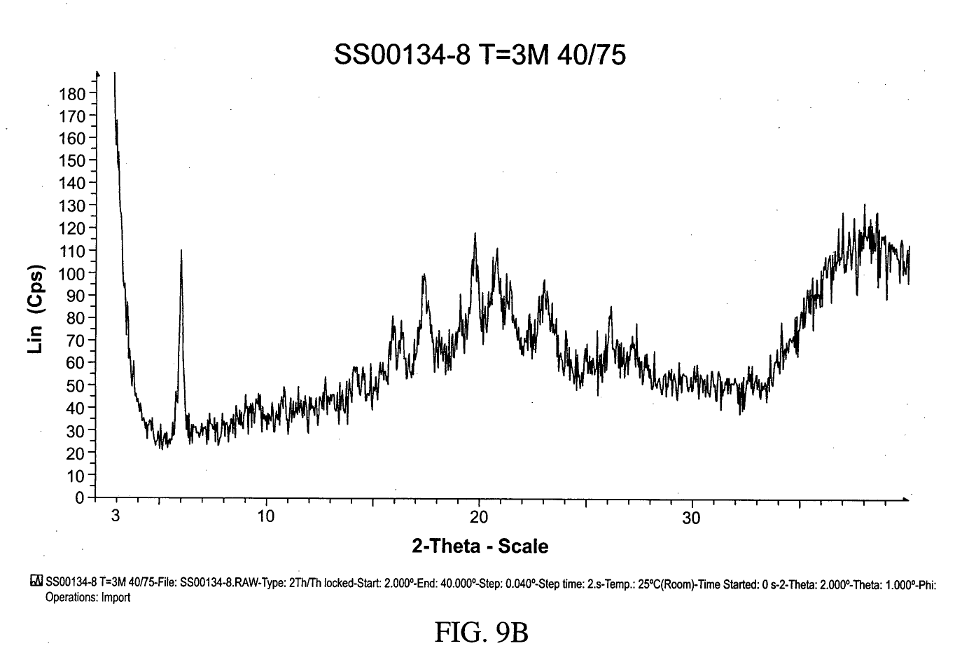

The XRD diffraction pattern of FIG. 9A showed that the product of Example B exhibits an amorphous XRD pattern which contains both an amorphous halo, and peaks, attributable to crystalline itraconazole. Since it is clear from the SEM images (see, for example FIG. 7A) that the itraconazole starting material was in crystalline form, the amorphous excipient is contributing significantly to the XRD pattern of the co-formulation. Samples were stored (in the form of the as-prepared powder) at 40° C. and 75% relative humidity in capped vials. The co-formulation is shown to be stable (that is, the extent of crystallinity remains constant) after storage for three months under these conditions (see FIG. 9B).

The following process examples illustrate various embodiments of the process for making the formulations of the present invention. All examples used the Nektar™ SCF particle precipitation process of the type described herein and/or in WO-03/008082 and/or WO-02/38127, and/or WO-01/15664. Process parameters described below include variations in: itraconazole:polymer ratios; internal vessel temperature; operating pressure; solution concentration; solution flow rate; and vessel size. Each resulting product was dissolution tested as described below. The polymer used was HPMC, as Pharmacoat™ 603. Several examples, as noted, were analyzed using XRD or XRPD.

### Process Example 1: Formulations Produced at Various Itraconazole:Polymer Ratios

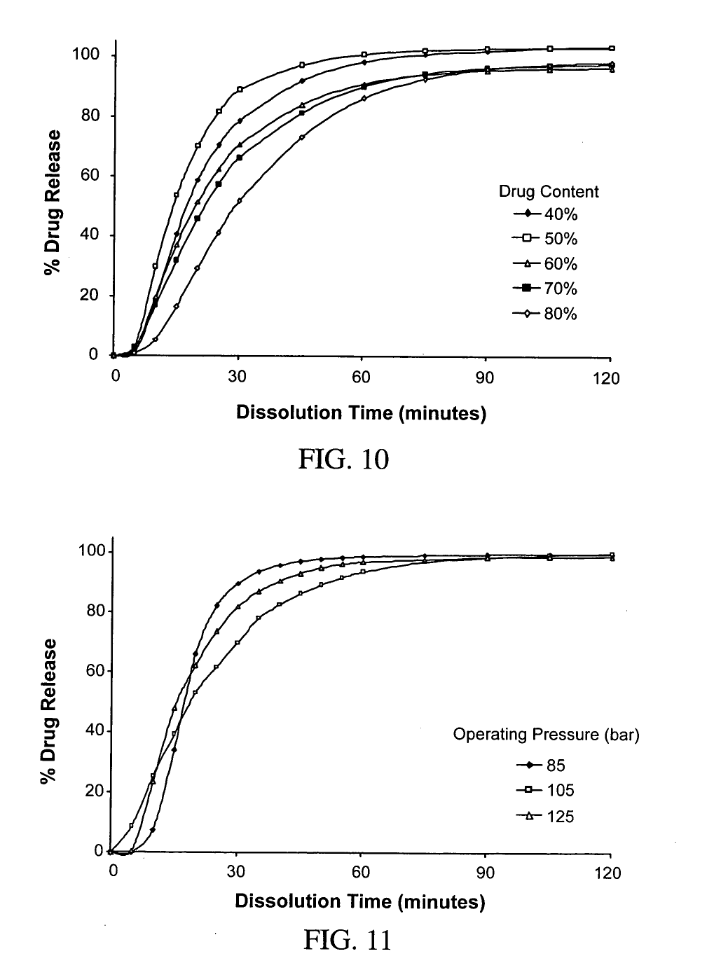

A range of formulations were processed to investigate the effect of increasing the ratio of itraconazole to HPMC in intervals of 10%, starting from 40% (w/w) to 80% (w/w) drug:polymer. Process parameters were: internal vessel temperature was 37° C., operating pressure was 85 bar; process solution concentration was 2.5% (w/v); process solution flow rate was 4 mL/min; CO₂ flow rate was 12-12.5 Kg/hr, and a 2 litre vessel was used. All process solutions were dissolved in a combination of methanol and dichloromethane in the ratio of 1:1 (v/v). Dissolution results are shown in FIG. 10. In the Fig., dissolution times for all ranges of drug:polymer are acceptable; the 50:50 ratio exhibits the fastest dissolution rate and shortest time to achieve about 94%, preferably about 95% and more preferably about 99% release of the itraconazole.

### Process Example 2: Formulations Produced at Various Operating Pressures

A range of formulations were processed to investigate the effect of increasing the operating pressure in intervals from 85 to 125 bar. Process parameters were: internal vessel temperature was 45-46° C., operating pressure was varied in 20 bar intervals between 85 and 125 bar; process solution concentration was 6.25% (w/v); process solution flow rate was 12 mL/min; CO₂ flow rate was 12-12.5 Kg/hr, and a 2 litre vessel was used. The formulation comprised 60% (w/w) itraconazole and 40% (w/w) HPMC dissolved in a combination of methanol and dichloromethane in the ratio of 1:1 (v/v).

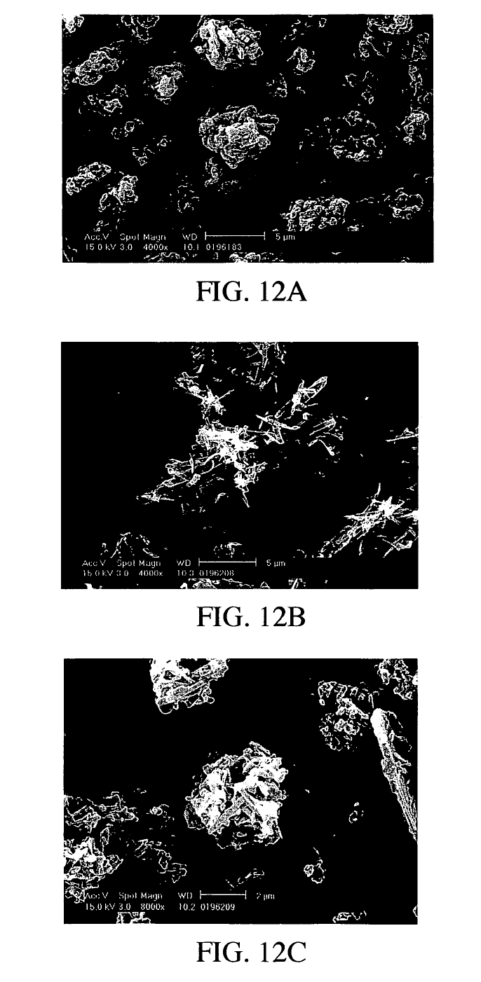

The dissolution performance of the various formulations is presented in FIG. 11. The dissolution rates for the products formed at all process pressures are acceptable in accordance with at least one object of the present invention. The 85 bar pressure conveys the fastest dissolution rate and the shortest time to achieve about 94%, preferably about 95% and more preferably about 99% release of the drug. FIGS. 12A, 12B and 12C are scanning electron micrographs of the formulations produced at 85 bar, 105 bar and 125 bar operating pressure respectively, and show resulting particle morphologies. FIG. 12A at 85 bar, illustrates the most preferred morphology wherein the amorphous polymer interacts most intimately with the crystalline drug. FIG. 12B at 125 bar is a less preferred morphology, showing large crystalline itraconazole “needles” and less drug/polymer interaction. FIG. 12C at 105 bar is a preferred morphology, and shows smaller crystalline “needles” with moderate interaction between drug and polymer.

### Process Example 3: Formulations Produced at Various Internal Vessel Temperatures

A range of formulations were processed to investigate the effect of increasing the internal vessel temperature in intervals from 36 to 50° C. Process parameters were: internal vessel temperature was varied, as indicated, between 36° C. and 50° C.; operating pressure was 95 bar; process solution concentration was 6.25% (w/v); process solution flow rate was 12 mL/min; CO₂ flow rate was 12-12.5 Kg/hr, and a 2 litre vessel was used. The formulation comprised 60% (w/w) itraconazole and 40% (w/w) HPMC dissolved in a combination of methanol and dichloromethane in the ratio of 1:1 (v/v).

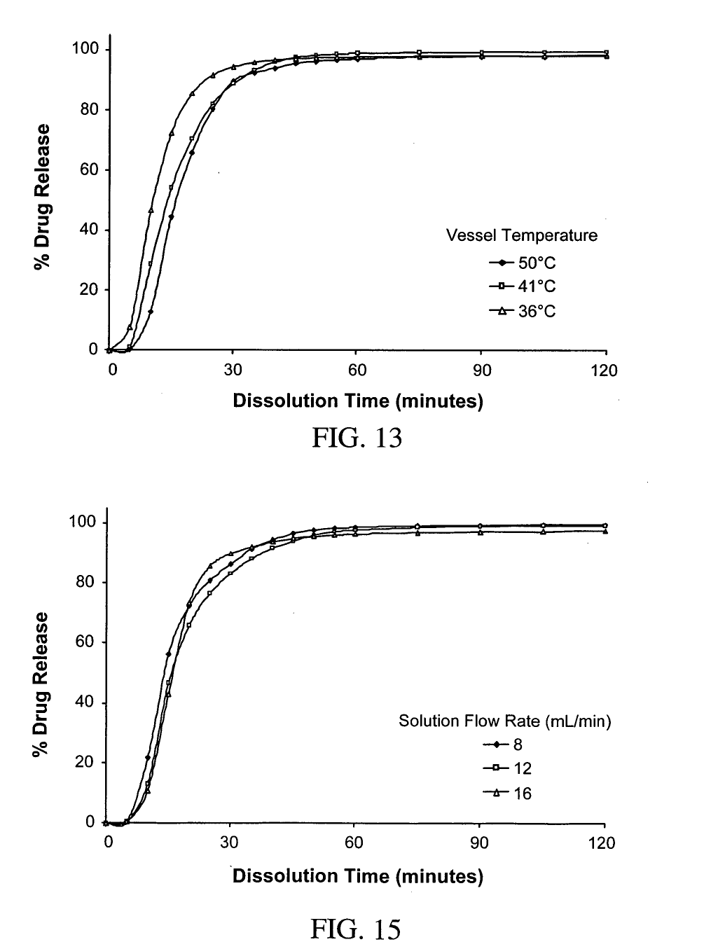

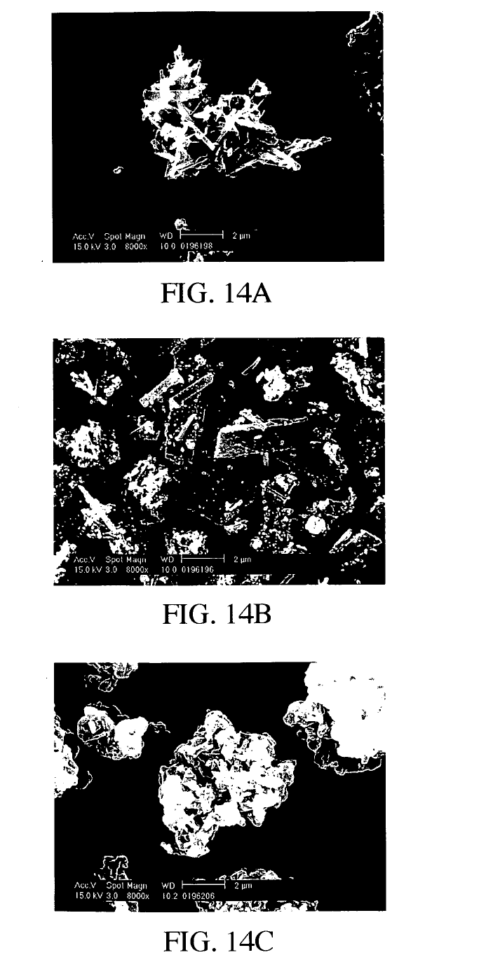

The dissolution performance of the various formulations is presented in FIG. 13. FIGS. 14A, 14B and 14C are scanning electron micrographs of the formulations produced at 36° C., 41° C., and 50° C. internal vessel temperatures respectively. From FIG. 13, it can be seen that the 36° C. temperature resulted in the formulation with the fastest dissolution rate; however, all temperatures tested yielded excellent dissolution rate results. In general, a higher process temperature provides a favored product morphology, but overall dissolution rates are comparable at these temperatures.

### Process Example 4: Formulations Produced at Various Process Solution Flow Rates

A range of formulations were processed to investigate the effect of increasing the process solution flow rate in intervals from 8 to 16 mL/min. Process parameters were: Internal vessel temperature was 47-49° C.; operating pressure was 95 bar; process solution concentration was 6.25% (w/v); process solution flow rate was varied, in 4 mL/min increments, between 8 and 16 mL/min; CO₂ flow rate was 12-12.5 Kg/hr, and a 2 litre vessel was used. The formulation comprised 60% (w/w) itraconazole and 40% (w/w) HPMC dissolved in a combination of methanol and dichloromethane in the ratio of 1:1 (v/v).

The dissolution performance of the various formulations is presented in FIG. 15. All three formulations displayed preferred dissolution curves. Scanning electron micrographs of the formulations produced at 8 mL/min, 12 mL/min, and 16 mL/min process solution flow rates all showed the preferred product morphology, that is, an intimate mixing of crystalline drug and amorphous polymer, with concomitant excellent dissolution and solubility results.

### Process Example 5: Formulations Produced at Various Process Solution Concentrations

A range of formulations were processed to investigate the effect of increasing the process solution concentration in intervals from 5.5% (w/v) to 7.0% (w/v). Process parameters were: internal vessel temperature was 47-48° C.; operating pressure was 95 bar; process solution concentration was varied, in indicated intervals, between 5.5 and 7.0% (w/v); process solution flow rate was 12 mL/min; CO₂ flow rate was 12-12.5 Kg/hr, and a 2 litre vessel was used. The formulation comprised 60% (w/w) itraconazole and 40% (w/w) HPMC dissolved in a combination of methanol and dichloromethane in the ratio of 1:1 (v/v).

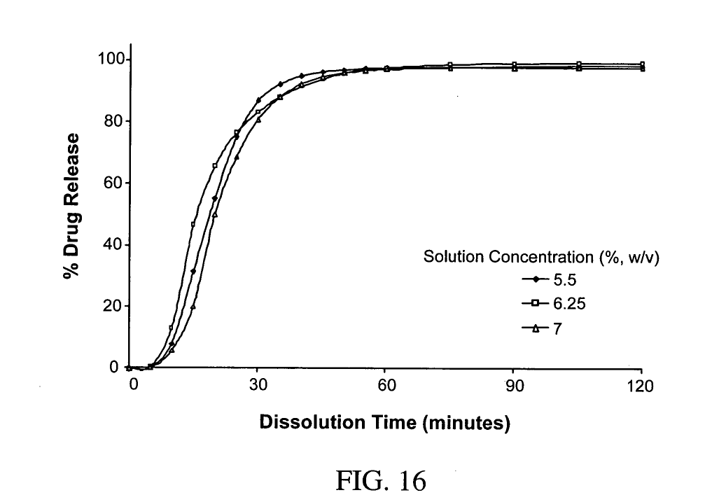

The dissolution performance of the various formulations is presented in FIG. 16. All three formulations displayed preferred dissolution curves. Scanning electron micrographs of the formulations produced at 5.5% (w/v), 6.25% (w/v), and 7.0% (w/v) process solution concentrations all showed the preferred product morphology, that is, an intimate mixing of crystalline drug and amorphous polymer, with concomitant excellent dissolution and solubility results.

### Process Example 6: Formulations Produced at Two Manufacturing Scales

Formulations were processed at a 2 Litre vessel and 10 Litre vessel to investigate the effect of process scale-up. Process parameters were: internal vessel temperature was 48° C.; operating pressure was 95 bar; process solution concentration was 6.25% (w/v); and the formulation comprised 60% (w/w) itraconazole and 40% (w/w) HPMC dissolved in a combination of methanol and dichloromethane in the ratio of 1:1 (v/v).

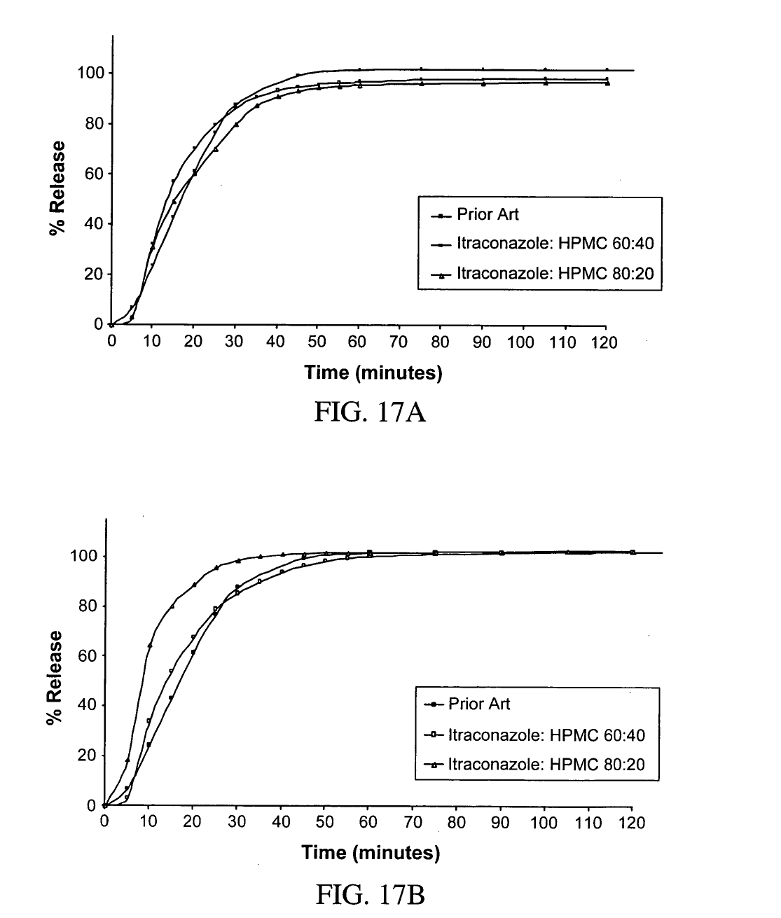

In one example, a 2 liter vessel was used, with a process solution flow rate of 8 mL/min and a CO₂ flow rate was 12-12.5 Kg/hr. In the other example, a 10 litre vessel was used, with a process solution flow rate of 2.1 kg/hr and a CO₂ flow rate of 50 Kg/hr. The dissolution performance of the various formulations made in the 2 litre vessel, compared to a prior art formulation comprising Sporanox™ capsules is presented in FIG. 17A. The dissolution performance of the various formulations made in the 10 litre vessel, compared to a prior art formulation comprising Sporanox™ capsules is presented in FIG. 17B. It can be seen that both the 2 and 10 liter vessel sizes resulted in a product with preferred dissolution characteristics.

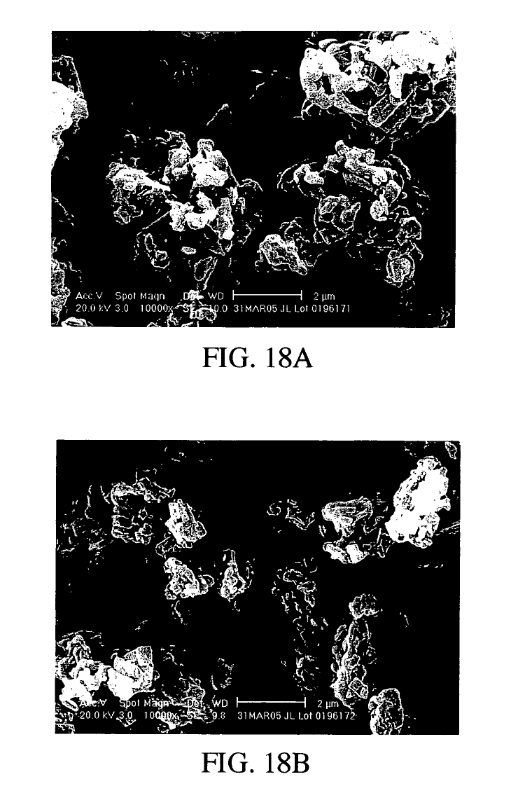

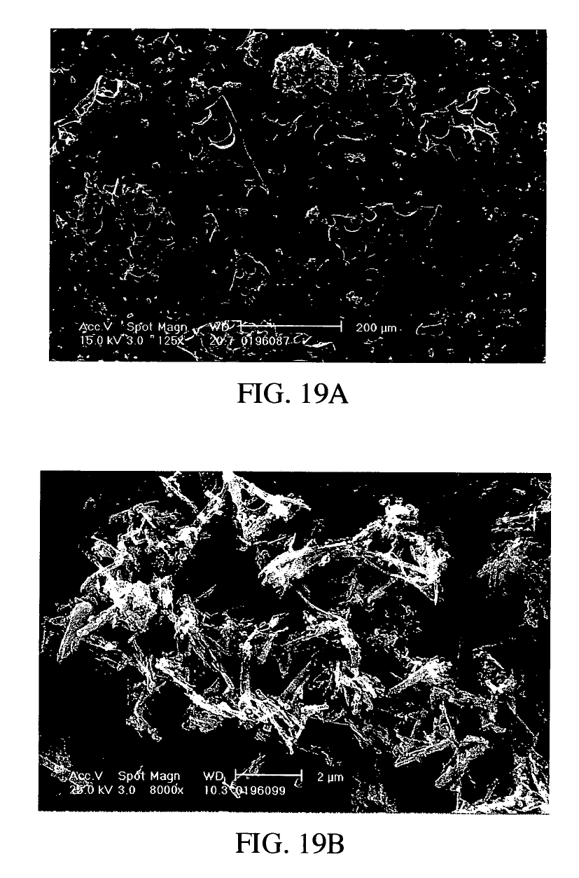

FIGS. 18A and 18B are SEM images of itraconazole produced using a co-formulation process of the present invention and wherein itraconazole is co-formulated with GMP compliant HPMC as Pharmacoat 603-NF. FIG. 18A shows a 60:40 ratio of itraconazole:HPMC, and FIG. 18B shows an 80:20 ratio of itraconazole:HPMC. It can be seen that the morphology of both formulations is similar, both showing the preferred product morphology, that is, an intimate mixing of crystalline drug and amorphous polymer, with concomitant excellent dissolution and solubility results, and bioavailability characteristics. FIGS. 19A and 19B are SEM images of itraconazole produced using a using the GAS particle precipitation method co-formulation process of the present invention and wherein itraconazole is co-formulated with PVP (FIG. 19A) and HPMC (FIG. 19B). Again, both images illustrate the predominance of the amorphous excipient, leading to the desired dissolution, solubility and bioavailability characteristics.

Dissolution Testing of Examples 1-6

Formulations were filled into gelatine capsules to give a total drug loading of 100 mg. The dissolution medium was BP artificial gastric fluid containing 1% (w/v) SDS and maintained at 37° C. Dissolution was performed using a Type II dissolution system as described below, using a stirring rate of 100 rpm with on line UV detection at 285 nm.

In Vivo Studies

An in vivo (rat) study was conducted. The release of a drug from an insoluble matrix can be modeled as a square root of a time-dependent process based on Fickian diffusion (R=k*t^{0.5}). When this model was applied to the data obtained, a good correlation between the rate of in vitro dissolution and the rate of in vivo absorption in rats was demonstrated. Such a correlation allows the dissolution method described herein to be used as a predictive screening tool during formulation development.

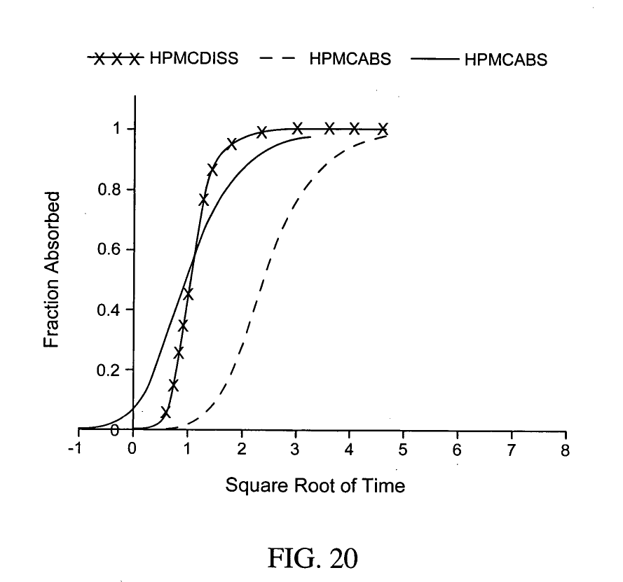

It is known to the art that a correlation exits between the rate of itraconazole dissolution in vitro and the rate absorbed in vivo (based upon human data). From published human data, it is clear that the amount dissolved at one hour, in an in vitro study correlates well with amount absorbed in vivo. FIG. 20 is a graph of fraction absorbed v square root of time (animal data, adjusted for an in vivo absorption lag) for a co-formulation of the present invention comprising itraconazole:HPMC in a 1:1 ratio. The solid line reflects the actual absorption data, while the line marked by the crosses represents an adjustment of the absorption data to remove the initial lag in comparing in vivo absorption with in vitro dissolution. This data may be correlated with standard methods of assessing bioavailability.

Analytical Techniques

The following analytical techniques were employed in the examples.

X-ray Powder Diffraction (XRPD)

The nature of the sample was characterized by XRD. An amorphous sample is indicated by the lack of diffraction peaks in the diffraction pattern which is characteristic of crystalline materials. Samples were analyzed (on a D5000 XRD (Siemens, Germany) between 2 and 40° 2θ, at a scan rate of 0.02 degrees per second.

Scanning Electron Microscopy (SEM)

Particle size and morphology were investigated using a FEI XL30 TMP Scanning Electron Microscope.

UV spectrophotometry. The weight fraction of drug in samples was measured with an Ultrospec™ 4000 spectrophotometer (Pharmacia Biotech, Cambridge, England), from reconstituted solutions of the samples. The absorbance of the polymers was negligible at the wavelengths used.

Dissolution Testing of Process Examples 1-6

Dissolution testing was carried out using an eight bath Copley dissolution kit with attached UV Spectrophotometer. The apparatus comprises an Erweka DT800 low head dissolution tester; an Ismatec IPC 8-channel peristaltic pump; a Perkin Elmer Lambda25 Spectrophotometer with cell changer and an Erweka fraction collector. The system is PC-controlled, via a Dissobox control unit.

The medium used was prepared from 1% SDS in BP Artificial Gastric Fluid): 20 g of sodium chloride, 32 g of pepsin powder, 78.4 ml of 10.2 Molar hydrochloric acid (1.16 specific gravity) and 100 g sodium dodecyl sulphate made up to 10 litres with deionised water. Dissolution was monitored for up to six hours at a constant temperature of 37° C.

Although the present invention has been described in considerable detail with regard to certain preferred versions thereof, other versions are possible. Accordingly, alterations, permutations and equivalents thereof will become apparent to those skilled in the art upon a reading of the specification and study of the drawings. Also, the various features of the embodiments herein can be combined in various ways to provide additional embodiments of the present invention. Furthermore, certain terminology has been used for the purposes of descriptive clarity, and not to limit the present invention. Therefore, any claims associated with the present disclosure should not be limited by the description of the preferred versions contained herein and should include all such alterations, permutations, and equivalents as fall within the true spirit and scope of the present invention.

1. A formulation comprising an azole antifungal and an excipient, the formulation prepared by a method comprising
providing a target solution comprising solution or suspension of azole antifungal; and
contacting the target solution with a compressed fluid anti-solvent under conditions which allow the anti-solvent to extract fluid from the target solution so as to cause particles of the formulated azole antifungal and excipient to precipitate.

2. The formulation of claim 1 wherein the anti-fungal comprises itroconazole and the excipient comprises an oligomer or polymer.

3. The formulation of claim 2 wherein the excipient comprises a hydrophobic polymer.

4. The formulation of claim 2 wherein, the oligomeric or polymeric excipient comprises a hydroxyalkylmethylcellulose, a viny pyrrolidone, or a combination thereof.

5. The formulation of claim 4, wherein a ratio of itraconazole to excipient ranges from about 40:60 to 80:20, inclusive.

6. The formulation of claim 4, wherein a ratio of itraconazole to excipient is about 1:1.

7. The formulation of claim 4 wherein the itraconazole is present in crystalline form.

8. The formulation of claim 4 wherein the itraconazole is present in non-crystalline form.

9. The formulation of claim 1 wherein the formulation is particulate, and is prepared by a gas anti-solvent precipitation method.

10. The formulation of claim 1 wherein the resulting material exhibits a dissolution characteristic substantially as shown in FIG. 16.

11. The formulation of claim 1 characterized in that the formulation exhibits at least one of; a 91% release or greater within 45 minutes; a physical stability or chemical stability of at least 3 months; a bulk density of at least about 0.9 g/ml; a tap density of at least about 0.12 g/ml and a release percentage of at least about 91% after 45 minutes.

12. The formulation of claim 1 wherein the formulation exhibits a morphology substantially as shown in FIG. 18.

13. The formulation according to claim 1, wherein the azole antifungal is crystalline, and is stable, with respect to reversion to its crystalline form(s), for at least three months following its formulation.

14. The formulation according to claim 1, which has been made by co-precipitating the azole antifungal and the excipient from a common solvent or solvent mixture using a compressed fluid anti-solvent.

15. The formulation according to claim 14, wherein the azole antifungal comprises itraconazole, and wherein the process further comprises contacting the target solution with a compressed fluid anti-solvent under conditions which allow the anti-solvent simultaneously both to disperse the target solution and to extract the vehicle from it so as to cause particles of itraconazole and excipient to precipitate as a co-formulation.

16. The formulation according to claim 15 wherein the excipient is HPMC.

17. The formulation according to claim 15 wherein the anti-solvent comprises CO₂, and the solvent comprises a hydroxylic solvent.

18. A pharmaceutical or nutraceutical composition comprising a formulation according to claim 1.

19. A product according to claim 18, comprising the composition, contained within a size 0 capsule intended for oral consumption.

20. The product according to claim 19 wherein the composition is chemically and physically stable for at least three months.

21. The product according to claim 19 wherein the composition exhibits a release percentage of at least about 91% after about 45 minutes.

22. A particulate co-formulation comprising an itraconazole and excipient, the co-formulation prepared by a gas anti-solvent precipitation method comprising
providing a target solution comprising solution or suspension of itraconazole and excipient in at least one fluid; and
contacting the target solution with a compressed fluid anti-solvent under conditions which allow the anti-solvent to simultaneously both to extract the target solution from the fluid, and to disperse the target solution it so as to cause particles of the coformulated itraconazole and excipient to precipitate from the fluid.

23. The co-formulation of claim 22 wherein the excipient is a hydrophobic polymer.

24. The co-formulation of claim 23 wherein, the polymer comprises a hydroxyalkylmethylcellulose, a viny pyrrolidone, or a combination thereof.

25. The co-formulation of claim 24, wherein a ratio of itraconazole to polymer ranges from 40:60 to 80:20, inclusive.

26. The co-formulation of claim 24, wherein a ratio of itraconazole to polymer is about 1:1.

27. The co-formulation of claim 22 wherein the itraconazole comprises its crystalline form.

28. The co-formulation of claim 22 wherein the itraconazole comprises its non-crystalline form.

29. The co-formulation of claim 22 characterized in that the co-formulation exhibits a 94% release or greater within 45 minutes, and a physical stability, or chemical stability, or both, of at least 3 months.

30. The co-formulation of claim 22 wherein the fluid used to extract the target suspension comprises CO₂.

31. The co-formulation of claim 22 wherein the co-formulation comprises an easy-flowing, non-cohesive, dispersed particulate powder.

32. The co-formulation of claim 31 wherein the co-formulation has at least one property selected from the group consisting of; a bulk density of at least about 0.9 g/ml; a tap density of at least about 0.12 g/ml and a release percentage of at least about 91% after 45 minutes.

33. A method of preparing a particulate co-formulation comprising itraconazole, the method comprising:
providing a particle formation vessel;
providing a solution or suspension of itraconazole and an excipient in a solvent; and
contacting the solution or suspension of itraconazole and excipient, within the particle formation vessel with a compressed fluid anti-solvent under at least one processing condition which allows the anti-solvent to extract fluid from the solution or suspension thereby causing the formation of particles comprising itraconazole and excipient.

34. The method of claim 33 wherein the excipient comprises an oligomer, a polymer, or a combination thereof.

35. The method of claim 33 wherein the excipient comprises HPMC.

36. The method of claim 33 wherein the excipient comprises PVP.

37. The method of claim 33 wherein a ratio of itraconazole to excipient is between 60:40 and 80:20, inclusive.

38. The method of claim 33 wherein the solvent comprises a hydroxylic solvent.

39. The method of claim 38 wherein the solvent comprises a mixture of dichloromethane and methanol.

40. The method of claim 33 wherein said at least one processing condition is selected from an operating pressure of between about 85 and 125 bar; an operating temperature of between about 36° C. and 50° C.; a process solution flow rate of between about 8-16 ml/min; a process solution concentration between about 5 and 7 weight percent; and combinations thereof.

41. The method of claim 33 wherein the anti-solvent fluid enters the particle formation vessel at a velocity of between about Mach 0.8 and 1.5; and the anti-solvent comprises supercritical CO₂.

42. A method for preparing a particulate co-formulation of itraconazole and an excipient, which involves co-precipitating the itraconazole and the excipient from target solution in a fluid vehicle by contacting the target solution with a compressed fluid anti-solvent under conditions which allow the anti-solvent simultaneously both to disperse the target solution and to extract the vehicle from it so as to cause particles of the coformulated itraconazole and excipient to precipitate.

43. A particulate co-formulation of itraconazole and an excipient produced by the method of claim 42.

44. A pharmaceutical composition comprising the particulate co-formulation of claim 42.

45. A pharmaceutical composition consisting essentially of an azole antifungal and an excipient, wherein the composition has a bulk density of at least about 0.9 g/ml, a tap density of at least about 0.12 g/ml, a release percentage of at least about 91% or more after 45 minutes, and is stable, as measured by x-ray diffraction or differential scanning calorimetry, for at least one month.

46. A formulation of crystalline itraconazole and a polymeric or oligomeric excipient having a morphology substantially as shown in FIG. 18.

47. The formulation of claim 46 comprising a particulate co-formulation having a bulk density of at least about 0.9 g/ml, a tap density of at least about 0.12 g/ml, a release percentage of at least about 91% or more after 45 minutes, and is stable, as measured by x-ray diffraction or differential scanning calorimetry, for at least one month.
